(19) United States
(12) Patent Application Publication
(10) Pub. No.: US 2003/0186223 A1
(43) Pub. Date: Oct. 2, 2003

(54) Modular recombinatorial display libraries

(75) Inventor: Robert C. Ladner, Ijamsville, MD (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Dyax Corp., Cambridge, MA

(21) Appl. No.: 10/378,557

(22) Filed: Mar. 3, 2003

Related U.S. Application Data
(60) Provisional application No. 60/361,121, filed on Mar. 1, 2002.

(51) Int. Cl. C12Q 1/70; C12Q 1/68

(52) U.S. Cl. 435/5; 435/6

(57) ABSTRACT

Modular display libraries are disclosed characterized by a display having two or more variable regions connected with constant regions encoded by DNA segments that include a restriction endonuclease cleavage site. The segments encoding modular variable regions can be recombined in anther position in the display vector or rearranged to extend the diversity of the original modular library.

### RELATED APPLICATIONS

This application claims the benefit of U.S. Provisional Application No. 60/361,121, filed on Mar. 1, 2002. The entire teachings of the above application are incorporated herein by reference.

### BACKGROUND OF THE INVENTION

Libraries of potential binders, particularly phage display libraries such as those described, for example, in Ladner et al., U.S. Pat. No. 5,223,409 and Kay et al., Phage Display of Peptides and Proteins: A Laboratory Manual (Academic Press, Inc., San Diego 1.996), provide a powerful tool for isolating binding polypeptides for a target molecule. In a phage display library, a variegated polypeptide coding sequence, fused with a coat protein gene of a recombinant phage, causes the variegated segment to be expressed as part of the phage particle surface, i.e., “displayed” on the phage surface of a large fraction of the phage particles. From such a library one can then select peptides that specifically bind a target molecule. Analysis of the sequences of selected variegated polypeptide display genes identifies a panel of target-binding polypeptides.

Phage display libraries typically contain from 10⁶ to 10¹⁰ different variegated polypeptides, and selecting from such a library against a target molecule can provide several dozen, hundreds, or even thousands of binders to the target. Where binders having special properties are sought, however, such as binders having especially high affinity for a target (e.g., having a dissociation constant, K_{D}, below 1 μM or even in the 1-50 nM range) or low off-rates (K_{off}) for a given target, a display library including over a billion unique sequences might yield only a few selectants having the desired properties. In such cases, the size of the display library, even though providing millions or hundreds of millions of potential binders, becomes a limiting factor in isolating a successful, specialized binding partner for a specific target.

To expose a given target to greater numbers of different potential binding polypeptides, several approaches have been proposed. For instance, larger libraries can be attempted, so that the original library will contain a greater number of permutations on a variable sequence and have a greater chance of producing a binding polypeptide of the desired characteristics. Alternatively, one or more promising binders isolated from an original library can be used as the basis for a directed or secondary library, e.g., by generating a library of analogues using the isolated binder as the parental template and allowing variegation at a few residues while holding other residues constant. Such a process is known as “affinity maturation”. Affinity maturation usually requires analysis of isolate sequences, selection of one or more parental sequences (i.e., to use as a secondary template), and synthesis of variegated DNA based on the selectant (template) to provide diversity around the selectant sequence in a secondary library. Another possibility for building a secondary library is to amplify one or more successful isolates from the original library using error-prone PCR, which will randomly variegate the selected sequences.

### SUMMARY OF THE INVENTION

The present invention provides another approach to library extension, in which display polypeptides are composed of two or more variegated modules, separated by constant regions encoded by DNA segments that include a restriction site. Successful binding modules can be swapped into the original library vector or recombined among themselves to form a secondary library providing additional levels of diversity from the variegation of the original library.

Accordingly, the present invention provides a population or library of display vectors comprising a multiplicity of DNA molecules comprising a general structure: R1-Z-R2, wherein R1 and R2 are, independently, variable regions of at least 3 bases (1 codon), wherein at least 3 bases are variable, and wherein Z is a constant region of at least 6 bases that includes the cleavage site of a restriction endonuclease. In preferred embodiments, the R1-Z-R2 cassette is bounded by restriction sites that are useful for manipulating the display vector. An enzyme is useful for manipulation if it cuts at a single site or if there are two sites, one in the Z region and one elsewhere. Preferably, the enzymes that cut these bounding restriction sites give cohesive ends that contain two, three, four, or more unpaired bases. Preferably, one or both of the cohesive ends are non-palindromic. Preferably, one of the restriction enzymes that cut at a bounding site gives a 3′ overhang and the other gives a 5′ overhang. In preferred embodiments, R1 and R2 are, independently, variable regions of at least 9 bases (3 codons), wherein at least 6 bases are variable, and wherein Z is a constant region of at least 6 bases that includes the cleavage site of a restriction endonuclease. More preferably, R1 and R2 are, independently, 9-36 bases, 12-36 bases, 15-36 bases, 9-24 bases, 12-24 bases, 15-24 bases, 9 bases, 12 bases, 15 bases, or 18 bases in length. Most preferably the variable (R) regions will be the same length. In particular embodiments, all the bases in variable regions R1 and R2 are variable (see, e.g., Example 4, infra), and in further embodiments, each of R1 and R2 contain 3 consecutive invariable bases, encoding cysteine (see, e.g., Examples 1 and 2, infra).

In preferred embodiments, the coding segment for the constant region, Z, is 6-36 bases, more preferably 6-9 bases. The restriction endonuclease cleavage site encompassed by Z is preferably one that produces non-palindromic cohesive ends. Preferred restriction enzymes, the recognition and cleavage sites for which can be utilized in designing the constant (Z) region include RsrII, BssSI, Bsu36I, AvaI, and the like. A particularly preferred example of a constant region useful in the present invention is 5′-tcCGGTCCG-3′ (hereinafter, “constant region 1”), which encodes a tripeptide Ser-Gly-Pro. The first two bases could be changed to give other amino acids in place of Ser. Constant region 1 contains an RsrII restriction-enzyme recognition site (RERS). Other preferred Z region sequences include 5′-CGGWCCG-3′, 5′-CTCGTG-3′, 5′-CACGAG-3′, and 5′-CYCCRG-3′, where if Y is C, R is A, and if Y is T, then R is G.

In a preferred embodiment, a library of vectors is provided comprising a multiplicity of DNA molecules having a general structure: R1-Z-R2, wherein R1 and R2 each encode a peptide of 7 amino acids having the formula: Xaa1-Xaa2-Xaa3-Cys-Xaa5-Xaa6-Xaa7, wherein each Xaa can be any amino acid except cysteine, and wherein Z encodes a tripeptide Ser-Gly-Pro. In preferred features, R1 comprises the sequence 5′-NNK NNK NNK TGY NNK NNK NNK-3′ and R2 comprises the sequence 5′-NNK NNK NNK TGY NNK NNK NNK-3′. Most preferably, said DNA molecules comprise the sequence: 5′-NNK NNK NNK TGY NNK NNK NNK TCC GGT CCG NNK NNK NNK TGY NNK NNK NNK-3′ (SEQ ID NO: 1).

As used herein, ‘NNK’ represents a variable codon that encodes all 20 encodable amino acids. NNK indicates that any base can be present at, for example, base 1, any base at base 2 and either G or T at base three. This provides three codons for Ser, Arg, and Leu; two codons for Gly, Ala, Val, Pro, and Thr; and one codon for each of Phe, Ile, Met, Tyr, His, Gln, Asn, Lys, Asp, Glu, Cys, Trp, and stop. A more preferred embodiment for these sequences and others in the present invention is one in which NNK represents a collection of codons such as {TTT, CTT, ATT, ATG, GTT, TCT, CCT, ACT, GCT, TAT, CAT, CAG, AAT, AAG, GAT, GAG, TGT, TGG, CGT, and GGT}, which encodes each amino acid once. For libraries of cyclic peptides, a most preferred embodiment for these sequences and others in the present invention is one in which NNK represents a collection of codons such as {TTT, CTT, ATT, ATG, GTT, TCT, CCT, ACT, GCT, TAT, CAT, CAG, AAT, AAG, GAT, GAG, TGG, CGT, and GGT}, which encodes each amino acid except cysteine once. Other choices of codons would work more or less as effectively. For example, change the Asp codon from GAT to GAC would be as effective.

In a further preferred embodiment, a library of vectors is provided comprising a multiplicity of DNA molecules having a general structure: R1-Z-R2, wherein R1 encodes a peptide having the sequence: Xaa1-Xaa2-Xaa3-Cys-Xaa5-Xaa6-Xaa7-Xaa8, wherein each Xaa can be any amino acid except cysteine; R2 encodes a peptide having the sequence: Xaa1-Xaa2-Xaa3-Xaa4-Cys-Xaa6-Xaa7-Xaa8, wherein each Xaa can be any amino acid except cysteine; and Z encodes a tripeptide, Ser-Gly-Pro.

In preferred features, said DNA molecules comprise the sequence: 5′-NNK NNK NNK TGY NNK NNK NNK NNK TCC GGT CCG NNK NNK NNK NNK TGY NNK NNK NNK-3′ (SEQ ID NO: 2).

Vectors suitable for construction of a library of bacteriophage M13 are provided by excising the NcoI/PstI digestion fragments of synthetic genes such as illustrated in FIGS. 1, 2, and 3 (SEQ ID NOS: 44, 46 and 48) and inserting the fragments into a suitable site in a phage display vector, such as the large fragment of NcoI/PstI digestion of the MANP vector depicted in FIGS. 4A-4C (SEQ ID NO: 57). An annotated version of MANP is given in FIGS. 5A-5F (SEQ ID NO: 58), including the cleavable signal sequence (SEQ ID NO: 59). FIGS. 4A-4C contains the unannotated DNA sequence of MANP (SEQ ID NO: 57). In MANP, the only copy of gene iii is modified so that there is an NcoI site in the end of the signal sequence followed by codons for BPTI and a linker, a PstI site, a linker that contains a factor Xa cleavage site, and the codons for mature III. Libraries are built by cutting MANP RF DNA with NcoI and PstI and ligating variegated DNA having the NcoI and PstI cohesive ends to the vector DNA. E. coli cells are transformed with the ligated DNA and these cells produce phage that display the encoded peptides. MANP also contains an ampicillin resistance gene (bla) obtained from pGEM3Zf and modified to remove unwanted restriction sites.

The present invention also provides a library of polypeptides comprised of the expression products of a library of vectors such as described above.

The present invention also provides a method for producing a modular phage display library comprising:

a) preparing a multiplicity of DNA molecules comprising a DNA sequence of the formula R1-Z-R2, wherein R1 and R2 are, independently, variable regions of at least 15 bases (5 codons) wherein at least 12 bases are variable, and wherein Z is a constant region of at least 6 bases that includes the cleavage site of a restriction endonuclease,

b) inserting said DNA molecules into phage display vector cassettes (e.g., such as MANP, FIGS. 4A-4C; SEQ ID NO: 57), and then

c) transfecting host bacteria with said phage display vector cassettes.

In preferred features, the phage display vector cassette includes at least one unique restriction endonuclease cleavage site. Preparation of such a modular display library provides a method of the present invention for producing a recombinatorial phage display library, comprising:

a) digesting phage display vector cassettes from the modular display library with said restriction endonuclease that cleaves within said constant region Z and a second restriction endonuclease that cleaves said vector cassette so as to yield a first and a second vector fragment, said first vector fragment including R1 and said second vector fragment including R2;

b) mixing said vector fragments together and religating to form recombinatorial phage vector cassettes; and

c) transfecting host bacteria with said recombinatorial phage vector cassettes.

Additional methods for producing alternative recombinatorial phage display libraries, e.g., secondary libraries utilizing coding segments for R1 and/or R2 that have been isolated by prior selection(s), are also contemplated and are described in detail infra.

The present invention also provides a library of recombinant bacteriophage displaying variegated polypeptides comprising the sequence: A1-B-A2, wherein A1 and A2 are, independently, variable region peptides of 5-12 amino acids, of which at least 4 amino acids are variable; and wherein B is a constant region peptide of 2-12 amino acids. In preferred embodiments, variable regions A1 and A2 are the same length. Preferably A1 and A2 are each about 6-9 amino acids, more preferably 7 or 8 amino acids in length. In particular embodiments, all the amino acid positions of the variable regions A1 and A2 are variable, and in further embodiments, each of the variable regions A1 and A2 contains an invariant cysteine residue (see, for example, Library MTN-13/I described in Example 2, infra). In preferred embodiments, the constant region, B, is 2-12 amino acids, more preferably 2-3 amino acids, most preferably 3 amino acids in length.

In a preferred embodiment, a modular library of polypeptides is produced, the polypeptides having the structure: A1-B-A2, wherein A1 and A2 are each a peptide of 7 amino acids having the sequence: Xaa1-Xaa2-Xaa3-Cys-Xaa5-Xaa6-Xaa7, wherein each Xaa can be any amino acid except cysteine, and wherein B encodes a tripeptide Ser-Gly-Pro. In a further preferred embodiment, a modular library of polypeptides is produced, the polypeptides having the structure: A1-B-A2, wherein A1 is a peptide of 8 amino acids having the sequence: Xaa1-Xaa2-Xaa3-Cys-Xaa5Xaa6-Xaa7-Xaa8, wherein each Xaa can be any amino acid except cysteine; A2 is a peptide of 8 amino acids having the sequence Xaa1-Xaa2-Xaa3-Xaa4-Cys-Xaa6-Xaa7-Xaa8, wherein each Xaa can be any amino acid except cysteine; and B encodes a tripeptide, Ser-Gly-Pro.

### BRIEF DESCRIPTION OF THE DRAWINGS

FIG. 1 illustrates a DNA sequence and corresponding amino acid sequence for a designed modular display library according to the invention. The synthetic 90-base oligonucleotide is provided with a 5′ NcoI restriction site and a 3′ PstI restriction site for excision and insertion into an NcoI-PstI-opened vector. The synthetic gene exhibits a display template having the structure of two 7-mer variable regions connected via a 3-amino acid constant region. The coding sequence for the constant region encompasses a restriction site for RsrII. The double underscored segments flanking the display template can be used to design primers for PCR amplification of the synthetic gene. As illustrated, the synthetic gene is designed for insertion into an M13 phage display vector, such that the display will be expressed, on propagation of the phage in E. coli, at the N-terminus of M13 protein III. The display template is designed with one invariant cysteine residue within each of the variable regions, so that on expression a cyclic microprotein will be formed and displayed on the phage in the library. Variegation of the amino acid positions shown as X to allow any amino acid except cysteine defines a library of 2.2×10¹⁵ possible sequences.

FIG. 2 illustrates the DNA sequence and corresponding amino acid sequence for a modular display library constructed in M13 phage in accordance with the invention (see Example 2). The synthetic 96-base oligonucleotide is provided with a 5′ NcoI restriction site and a 3′ PstI restriction site for excision and insertion into an NcoI-PstI-opened phage display vector. The synthetic gene exhibits a display template having the structure of two 8-mer variable regions connected via a 3-amino acid constant region (Ser-Gly-Pro). The coding sequence for the constant region encompasses a restriction site for RsrII. The double underscored segments flanking the display template were used to design primers for PCR amplification of the synthetic gene. The synthetic gene was designed for insertion into an M13 phage display vector, such that the display will be expressed, on propagation of the phage in E. coli, at the N-terminus of M13 protein III. The display template is designed with one invariant cysteine residue within each of the variable regions, so that on expression a cyclic microprotein will be formed and displayed on the phage in the library. Variegation of the amino acid positions shown as X to allow any amino acid except cysteine defined a library of 8.0×10¹⁷ possible sequences. Cleaving the isolated DNA with RsrII and another restriction enzyme having a unique site within the vector separates the two coding segments for the variable regions, permitting recombination to form new diversity not captured in the original library. DNA from phage isolated in initial rounds of screening can be cleaved and recombined with each other or with another collection of similarly cleaved DNA, such as the amplified DNA of the unselected library, thereby creating a secondary library of extended diversity compared to the original library. Such diversity extension is illustrated in Example 2. When the illustrated segment is inserted into M13 gene III, the amino acids S, M, A are the last three of the protein III signal sequence, and signal peptidase I cleaves just before A at position 1.

FIG. 3 illustrates a DNA sequence and corresponding amino acid sequence for a designed modular linear display library according to the invention. The synthetic 90-base oligonucleotide is provided with a 5′ NcoI restriction site and a 3′ PstI restriction site for excision and insertion into an NcoI-PstI-opened vector. The synthetic gene exhibits a display template having the structure of two 8-mer variable regions connected via a 3-amino acid constant region. The coding sequence for the constant region encompasses a restriction site for Bsu36I. The double underscored segments flanking the display template can be used to design primers for PCR amplification of the synthetic gene. As illustrated, the synthetic gene is designed for insertion into an M13 phage display vector, such that the display will be expressed, on propagation of the phage in E. coli, at the N-terminus of M13 protein III. The display template is designed so that on expression a linear polypeptide will be formed and displayed on the phage in the library. Variegation of the amino acid positions shown as X to allow any amino acid except cysteine defines a library of 2.9×10²⁰ possible sequences.

FIGS. 4A-4C set forth the nucleotide sequence for the MANP vector (Dyax Corp., Cambridge, Mass.), which is a suitable vector for creating an M13 phage display library in accordance with the present disclosure.

FIGS. 5A-5F set forth the annotated DNA sequence of MANP.

### DEFINITIONS

In the following sections, the term “recombinant” is used to describe non-naturally altered or manipulated nucleic acids, host cells transfected with exogenous nucleic acids, or polypeptides expressed non-naturally, through manipulation of isolated DNA and transformation of host cells. Recombinant is a term that specifically encompasses DNA molecules that have been constructed in vitro using genetic engineering techniques, and use of the term “recombinant” as an adjective to describe a molecule, construct, vector, cell, polypeptide, polynucleotide, or population (library) of polypeptides or polynucleotides specifically excludes naturally occurring such molecules, constructs, vectors, cells, polypeptides, polynucleotides, or libraries.

The term “bacteriophage” is defined as a bacterial virus containing a DNA core and a protective shell built up by the aggregation of a number of different protein molecules. The terms “bacteriophage” and “phage” are used herein interchangeably. Unless otherwise noted, the terms “bacteriophage” and “phage” also encompass “phagemids” (i.e., a plasmid that includes a portion of the genome of a bacteriophage so that the DNA can be packaged by coinfection of a phagemid-infected host with a helper phage) as well known by practitioners in the art. In preferred embodiments of the present invention, the phage is an M13 phage.

The term “polypeptide” is used to refer to a compound of two or more amino acids joined through the main chain (as opposed to side chain) by a peptide amide bond (-—C(:O)NH—). The term “peptide” is used interchangeably herein with “polypeptide” but is generally used to refer to polypeptides having fewer than 40, and preferably fewer than 25 amino acids.

The term “binding polypeptide” as used herein refers to any polypeptide capable of forming a non-covalent binding complex with another molecule. An equivalent term sometimes used herein is “binding moiety”. “KDR binding polypeptide” is a binding polypeptide that forms a complex in vitro or in vivo with vascular endothelial growth factor receptor-2 (or KDR). Specific examples of KDR binding polypeptides are illustrated in Tables 1, 2, and 3, infra.

The term “binding” refers to the determination by standard assays, including those described herein, that a binding polypeptide recognizes and binds reversibly to a given target. Such standard assays include equilibrium dialysis, gel filtration, surface plasmon resonance (SPR), and the monitoring of spectroscopic changes that result from binding.

The terms “homologous” or “homologue”, as used herein, refers to the degree of sequence similarity between two polymers (i.e., polypeptide molecules or nucleic acid molecules). When the same nucleotide or amino acid residue or one with substantially similar properties (i.e., a conservative substitution) occupies a sequence position in the two polymers under comparison, then the polymers are “homologous” at that position, and the polymers are referred to as “homologues”. For example, if the amino acid residues at 60 of 100 amino acid positions in two polypeptide sequences match or are homologous, then the two sequences are 60% homologous. The homology percentage figures referred to herein reflect the maximal homology possible between the two polymers, i.e., the percent homology when the two polymers are so aligned as to have the greatest number of matched (homologous) positions. Percent homology or percent identity of two amino acid sequences or of two nucleic acid sequences can be conveniently determined using the algorithm of Karlin and Altschul (Proc. Natl. Acad. Sci. USA, 87: 2264-2268 (1990)), modified as in Karlin and Altschul (Proc. Natl. Acad. Sci. USA, 90: 5873-5877 (1993)). Such an algorithm is incorporated into the NBLAST and XBLAST programs of Altschul et al. (J. Mol. Biol., 215: 403-410 (1990)). BLAST nucleotide searches can be performed with the NBLAST program to obtain nucleotide sequences homologous to a nucleic acid molecule described herein. BLAST protein searches can be performed with the XBLAST program to obtain amino acid sequences homologous to a reference polypeptide. To obtain gapped alignments for comparison purposes, Gapped BLAST is utilized as described in Altschul et al. (Nucleic Acids Res., 25: 3389-3402 (1997)). When utilizing BLAST and Gapped BLAST programs, the default parameters of the respective programs (e.g., XBLAST and NBLAST) are used. These BLAST programs are accessible on the worldwide web at ncbi.nlm.nih.gov.

The term “specificity” refers to a binding polypeptide having a higher binding affinity for one target over another. For example, the term “KDR specificity” would refer to a KDR binding moiety having a higher affinity for KDR over an irrelevant target. Binding specificity can be characterized by a dissociation equilibrium constant (K_{D}) or an association equilibrium constant (Kₐ) for the two tested target materials.

### DETAILED DESCRIPTION OF THE INVENTION

The present invention provides a novel approach to providing display libraries that can be efficiently recombined to provide increased diversity of displayed sequences, thereby increasing the potential of the library to yield a binding polypeptide having the characteristics desired. The library designs described herein feature a display template with two or more variable regions connected via a constant region of at least two amino acids, the coding segment for which encompasses a restriction enzyme cleavage site. Construction of the modular library using standard techniques in the art provides a display library having a degree of diversity corresponding to the degree of variegation of the variable positions of the variable regions. This diversity can be extended according to the invention by collecting the DNA of the display library or selectants from it and cleaving the DNA with restriction enzymes corresponding to the coding segment(s) for a constant region, which will separate variable region coding sequences. The separated variable region coding sequences can be mixed and recombined to provide combinations of variable regions that were not present in the original library and/or that combine a preferred selected variable region showing a particular affinity for a target with a range of additional variable regions, forming a large population of new polypeptides based on the selectants. This provides a means of affinity maturation of selected binding polypeptides that provides a vast number of homologous sequences, rather than being confined to point mutations at a few positions in a selected binding polypeptide.

A modular library according to the invention can have two, three, four, five, or more variable regions, each connected via a constant region. The coding sequence for the library must be designed such that at least one of the constant regions is encoded by a segment that is cleavable by a restriction enzyme, thereby permitting separation and recombination of a least two of the variable regions. The oligonucleotide sequence encoding a modular library according to the invention thus will have a formula: R-Z-R-Z-R-Z-R-, etc., wherein each R is a variable region and each Z is a constant region, and at least one Z region encompasses a cleavage site for a restriction endonuclease. Preferably, the Z region will encompass the recognition sequence of a restriction endonuclease, which endonuclease cleaves within the recognition sequence. Most preferably, the restriction endonuclease recognition sequence will be nine bases or fewer, and cleavage by the endonuclease will result in non-palindromic cohesive ends. Moreover, it is preferred that for each Z region encompassing a restriction site, the site will be a unique site within the library vector.

The restriction endonuclease recognition sequence of the Z region will preferably be as short as possible while still providing a unique site within the vector. Z regions of nine bases or fewer (i.e., encoding three amino acids or fewer) are most preferred. The longer the Z region, the more constant amino acids will separate the variable (R) regions. In general, it is preferred to have the Z region as short as possible and to have sequences that do not encode peptides that are non-specific binders, are insoluble, or are easily cleaved by proteases: For example, clusters of hydrophobic residues can make peptides insoluble, non-specifically adherent to cellular structures, or prone to micelle-formation in aqueous solution, therefore clusters of hydrophobic residues encoded by the constant (Z) region are not preferred. Also, many proteases cleave after Arg or Lys, therefore it is preferable that these residues are not encoded in the Z region, although when Arg or Lys are immediately followed by Pro, cleavage is usually blocked, hence Arg-Pro or Lys-Pro are as acceptable as are other peptide sequences. While it is preferable for the Z region to be as short as possible so as minimize separation between variable regions, the Z region should be distinct from, i.e., should not extend into, the flanking variable regions, as this limits variability and leads to incomplete recombination when the R modules are separated by cleaving the constant (Z) region.

As stated previously, it is also preferred that the restriction site of the Z region will produce non-palindromic cohesive ends. Many suitable restriction endonuclease recognition sequences of this type are known, including without limitation the recognition sequences of the following enzymes: AccI (GTMKAC), AflIII (ACRYGT), AlwNI (CAGNNNCTG), AvaI (CYCGRG), BanI (GGYRCC), BanII (GRGCYC), BlpI (GCTNAGC), BsaJI (CCNNGG), BsiEI (CGRYCG), BsiHKAI (GWGCWC), Bsp1286I (GDGCHC), BsrI (ACTGGNN), BsrI (NCCAGT), BsrDI (GCAATGNNN), BsrDI (NNCATTGC), BssSI (CTCGTG), BssSI (CACGAG), BstEII (GGTNACC), Bsu36I (CCTNAGG), DraIII (CACNNNGTG), DsaI (CCRYGG), EcoO109I (RGGNCCY), EspI (GCTNAGC), PpuMI (RGGWCCY), RsrII (CGGWCCG), SexAI (ACCWGGT), SfcI (CTRYAG), StyI (CCWWGG). In the foregoing list, the recognition sequence for the enzyme is shown in parentheses, with the cohesive ends indicated by underlining. Where the recognition sequence of the restriction enzyme accepts ambiguous bases in a symmetric arrangement, it is preferred that the choice of actual DNA sequence will render the cohesive ends non-palindromic. For example, SfcI recognizes CTRYAC, and it is preferred to use either CTACAC or CTGTAC but not CTATAC or CTGCAG.

EspI is an enzyme that could be used for manipulation if two sites are present. A preferred embodiment would be to have the sequence 5′-GCTCAGCct-3′ in the Z region and 5′-GCTAAGC-3′ elsewhere. EspI will cut both sites, but the ends will go together only in the desired manner.

Also, for the coding sequence for a modular library of this invention, the variable (R) regions preferably are equal or approximately equal in size, and preferably the constant (Z) region(s) are designed so as to promote interaction or cooperation, on expression, between two or more variable regions. For instance, the constant region can be designed to have flexibility to promote an ability of adjacent variable regions to bind simultaneously to a target. Alternatively, the constant region can be designed to have a particular configuration that places adjacent variable regions in a desired spatial relationship. For example, specific modular library designs described herein employ a tripeptide constant region, e.g., Ser-Gly-Pro or Pro-Ser-Gly, that are expected to cause a bend or a turn in the expressed amino acid sequence.

A modular library having two variable regions is illustrated by a template coding sequence of the formula R1-Z-R2, wherein R1 and R2 are, independently, variable regions of at least 3 bases (1 codon), wherein at least 3 bases are variable, and wherein Z is a constant region of at least 6 bases that includes the cleavage site of a restriction endonuclease. Thus, the variable R regions can advantageously be 3, 6, 9, 12, 15, 18, 21, 24, 27, 30, 33, 36, 39 or more bases in length, i.e., 1, 2, 3, 4, 5, 6, 7, 8, 9, 10, 11, 12, 13 or more codons in length, optionally with one or more (preferably one) invariant codons. It will be appreciated that very short variable regions of one or two or three codons will provide comparatively small libraries upon expression, and that long variable regions having above about eight variable codons will provide such immense diversity that only a very small fraction of the variegated sequences will ever be captured and sampled. Thus, in preferred embodiments, R1 and R2 are, independently, variable regions of at least 9 bases (3 codons), wherein at least 6 bases are variable, and wherein Z is a constant region of at least 6 bases that includes the cleavage site of a restriction endonuclease. More preferably, R1 and R2 are, independently, 9-36 bases, 12-36 bases, 15-36 bases, 9-24 bases, 12-24 bases, 15-24 bases, 9 bases, 12 bases, 15 bases, or 18 bases in length. Most preferably the variable (R) regions will be the same length. In particular embodiments, all the bases in variable regions R1 and R2 are variable (see, e.g., Example 4, infra), and in further embodiments, each of R1 and R2 contain 3 consecutive invariable bases, encoding cysteine (see, e.g., Examples 1 and 2, infra).

In a particularly preferred embodiment, a modular library having two variable regions comprises a template coding sequence R1-Z-R2, wherein R1 and R2 are, independently, variable regions of at least 15 bases (5 codons) wherein at least 12 bases are variable, and wherein Z is a constant region of at least 6 bases that includes the cleavage site of a restriction endonuclease. The template thus encodes a display polypeptide comprising a first variable region of at least 5 amino acids and at least 4 variable amino acid positions, a constant region of at least two amino acids, and a second variable region of at least 5 amino acids and at least 4 variable amino acid positions. In one preferred embodiment, one amino acid position in each variable region will be an invariant cysteine, and most preferably the variegation of the remaining amino acid positions will exclude cysteine. This leads to a library of display peptides capable of forming a cyclic structure involving both variable regions. In another preferred embodiment, no cysteines are allowed at any position.

The modular libraries described herein can be constructed for expression in any replicable genetic package, but phage or yeast display libraries are particularly preferred. The modular libraries will be described herein with reference to phage display, however it will be readily apparent to those skilled in the art that the principles described herein can easily be applied to other types of recombinant libraries, including display libraries or intracellular libraries. Modular libraries, especially linear modular libraries, are particularly applicable to “yeast two-hybrid” selection.

In order to prepare a modular library of phage display polypeptides to screen for binding polypeptides, a candidate binding domain is selected to serve as a structural template for the peptides to be displayed in the library. The phage library is made up of a multiplicity of analogues of the parental domain or template. The binding domain template can be a naturally occurring or synthetic protein, or a region or domain of a protein. The binding domain template can be selected based on knowledge of a known interaction between the binding domain template and the binding target, but this is not critical. In fact, it is not essential that the domain selected to act as a template for the library have any affinity for the target at all: Its purpose is to provide a structure from which a population (library) of similarly structured polypeptides (analogues) can be generated, which multiplicity of analogues will hopefully include one or more analogues that exhibit the desired binding properties (and any other properties screened for).

In selecting the parental binding domain or template on which to base the variegated amino acid sequences of the library, the most important consideration is how the variegated peptide domains will be presented to the target, i.e., in what conformation the peptide analogues will come into contact with the target. In phage display methodologies, for example, the analogues will be generated by insertion of synthetic DNA encoding the analogues into phage, resulting in display of the analogue on the surfaces of the phage. Such libraries of phage, such as M13 phage, displaying a wide variety of different polypeptides, can be prepared using techniques as described, e.g., in Kay et al., Phage Display of Peptides and Proteins: A Laboratory Manual (Academic Press, Inc., San Diego 1996) and U.S. Pat. No. 5,223,409 (Ladner et al.), incorporated herein by reference.

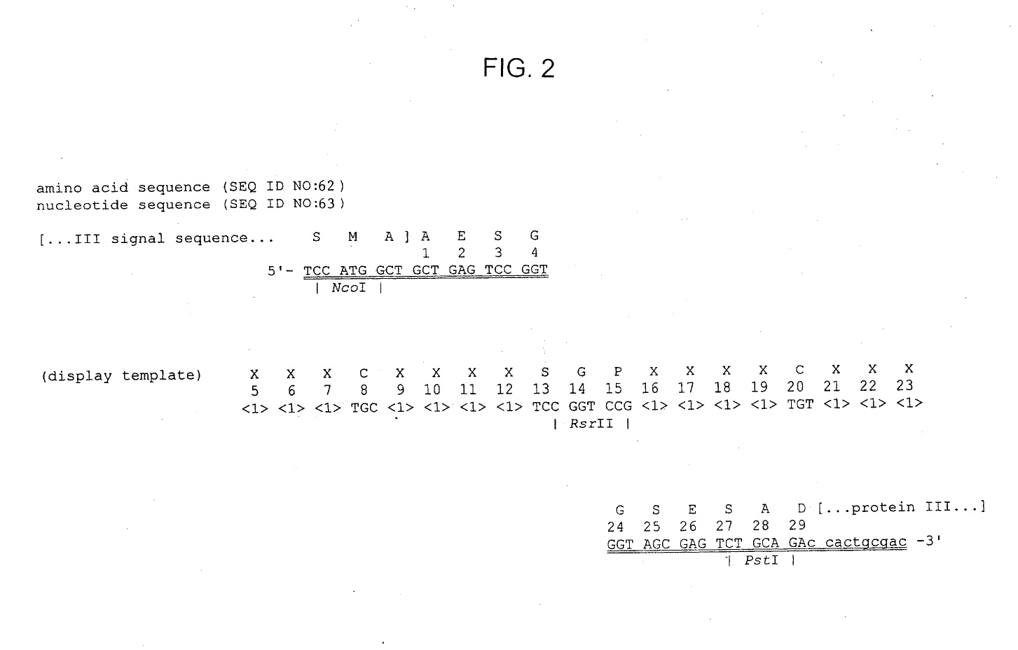

The construction of a phage display library having a template coding sequence of the formula R1-Z-R2 is described in Example 2 and illustrated in FIG. 2. The MTN-13/I library was constructed to display a single microprotein binding loop contained in a 19-amino acid template featuring two variable regions of equal size (i.e., eight amino acids) separated by a constant region of three amino acids (Ser-Gly-Pro). The MTN-13/I library utilized a template sequence 5′-NNK NNK NNK TGY NNK NNK NNK TCC GGT CCG NNK NNK NNK TGY NNK NNK NNK-3′ (SEQ ID NO: 1), which encoded a display polypeptide having the sequence Xaa1-Xaa2-Xaa3-Cys-Xaa5-Xaa6-Xaa7-Xaa8-Ser-Gly-Pro-Xaa12-Xaa13-Xaa14-Xaa15-Cys-Xaa17-Xaa18Xaa19 (SEQ ID NO: 3). The amino acids at positions 1, 2, 3, 5, 6, 7, 8, 12, 13, 14, 15, 17, 18, and 19 in the template were varied to permit any amino acid except cysteine (Cys). After expression of the MTN-13/I library in M13 phage, the library was screened to select binding peptides for a KDR target, and DNA from the selectants was cleaved to separate the variable region coding sequences, which was in turn recombined with similarly cleaved DNA from the unselected original library, to create a secondary library adding additional diversity to the selectants isolated against KDR target from the MTN-13/I library. Two rounds of selection of the secondary library revealed several unique high affinity KDR binding polypeptides.

Display libraries according to the invention can be created by making a designed series of mutations or variations within a coding sequence for the polypeptide template, each mutant sequence encoding a peptide analogue corresponding in overall structure to the template except having one or more amino acid variations in the sequence of the template. The novel variegated (mutated) DNA provides sequence diversity, and each transformant phage displays one variant of the initial template amino acid sequence encoded by the DNA, leading to a phage population (library) displaying a vast number of different but structurally related amino acid sequences. The amino acid variations are expected to alter the binding properties of the binding peptide or domain without significantly altering its structure, at least for most substitutions. It is preferred that the amino acid positions that are selected for variation (variable amino acid positions) will be surface amino acid positions, that is, positions in the amino acid sequence of the domains that, when the domain is in its most stable conformation, appear on the outer surface of the domain (i.e., the surface exposed to solution). Most preferably the amino acid positions to be varied will be adjacent or close together, so as to maximize the effect of substitutions.

As indicated previously, the techniques discussed in Kay et al., Phage Display of Peptides and Proteins: A Laboratory Manual (Academic Press, Inc., San Diego 1996) and U.S. Pat. No. 5,223,409 are particularly useful in preparing a library of potential binders corresponding to the selected or designed parental template. The MTN-13/I original and secondary libraries discussed herein were prepared according to such techniques, and they were screened for KDR binding polypeptides against an immobilized target, as explained in the examples to follow.

In a typical selection, a phage library is contacted with and allowed to bind the target, or a particular subcomponent thereof. To facilitate separation of binders and non-binders, it is convenient to immobilize the target on a solid support (e.g., magnetic beads). Phage bearing a target-binding moiety form a complex with the target on the solid support, whereas non-binding phage remain in solution and can be washed away only with excess buffer. Bound phage are then liberated from the target by changing the buffer to an extreme pH (pH 2 or pH 10), changing the ionic strength of the buffer, adding denaturants, or other known means. Alternatively, the binding phage need not be eluted at all but can be used intact in a complex with the target to infect host bacteria to propagate successful binders. To isolate the KDR binding phage from the MTN-13/I library, very high affinity binding phage that could not be competed off the immobilized target by incubating with VEGF overnight were captured by using the phage still bound to substrate for infection of E. coli cells.

The recovered phage can then be amplified through infection of bacterial cells and the screening process repeated with the new pool that is now depleted in non-binders and enriched in binders. The recovery of even a few binding phage is sufficient to carry the process to completion. After a few rounds of selection, the gene sequences encoding the binding moieties derived from selected phage clones in the binding pool are determined by conventional methods, described below, revealing the peptide sequence that imparts binding affinity of the phage to the target. When the selection process works, the sequence diversity of the population falls with each round of selection until desirable binders remain. The sequences converge on a small number of related binders, typically 10-50 out of the more than 100 million original candidates from each library. An increase in the number of phage recovered at each round of selection, and of course, the recovery of closely related sequences are good indications that convergence of the library has occurred in a screen. After a set of binding polypeptides is identified, the sequence information can be used to design other secondary phage libraries, biased for members having additional desired properties.

After one or more rounds of selection, the population of selected phage contains (a) phage that bind target due to a sequence in the first variable region, (b) phage that bind target due to a sequence in the second variable region, and (c) phage that bind target due to the sequences of both the first and second variable regions. Without sequencing or analyzing the selectants, one or more secondary libraries can be readily constructed that are likely to be rich in target binders. Examples of such secondary libraries include, but are not limited to, libraries in which:

1) the oligonucleotides encoding the first variable regions of the selectants are replaced with the oligonucleotides encoding the first variable region of the original (parental) library. This yields a library diversifying the (b) phage, pairing the entire repertoire of the parental library's first variable region with the second variable region of the selectants.

2) the oligonucleotides encoding the second variable regions of the selectants are replaced with the oligonucleotides encoding the second variable region of the original (parental) library. This yields a library diversifying the (a) phage, pairing the entire repertoire of the parental library's second variable region with the first variable region of the selectants.

3) the oligonucleotides encoding the first and second variable regions are excised and recombined within the selected pool. The forms novel combinations of variable regions from successful binders in all categories (a), (b) and (c).

4) the replicative form DNA (RF DNA) from the selected phage is digested with the restriction endonucleoase of the restriction endonuclease recognition site encompassed by the constant region and a second restriction endonuclease that cleaves the RF DNA in a different, unique site; the parental library is digested with the same two restriction enzymes; the DNAs are mixed in approximately equimolar amounts, religated and used to transform cells. This forms a library comprising approximately equal numbers of members having:

i) a selected first variable region and a library second variable region;

ii) a library first variable region and a selected second variable region;

iii) a selected first variable region and a selected second variable region;

iv) a library first variable region and a library second variable region.

Components (i), (ii) and (iii) of this library correspond to libraries (1), (2) and (3), above, respectively.

Selection for binding from any of these libraries allows isolation of sequences that were not present in the original library. For libraries having more than about eight variable positions, it is not efficacious to make all possible sequences and have them present in one library (e.g., 2.0×10¹⁰ different transformants for a variegated 8-mer permitting all possible amino acids at any position but excluding cysteine gives about 69% of the allowed 1.7×10¹⁰). Yet, high affinity binding often requires the proper positioning of more than eight amino acids. With the modular libraries of the present invention, the effect of screening a larger library is obtained from a smaller original library by recombining variable regions in secondary libraries of extended diversity. By way of illustration, if an original modular library with two variable regions contains 3×10⁹ sequences, and a first pool of selectants contains, e.g., 6×10³ isolates, it will usually be observed that among the isolates the sequences of the first variable region are paired with, in most cases, only one second variable region sequence (that is, there are very few early round isolates from the original library that have a first or second variable region that also appears in another isolate sequence). If a secondary library is now prepared according to procedure (4) above, a library having about 1.2×10⁹ members can be made, and each of the selected first variable regions will be now be paired with 3×10⁸ variants of the second variable region, each of the selected second variable regions will now be paired with 3×10⁸ variants of the first variable region, all possible combinations of the selected first and second variable regions will appear, and 3×10⁸ new unselected library sequences will appear. Thus, it can be calculated that selectants from this secondary library could only have been isolated from a naive (unselected original) library 10,000 times larger than the original library (e.g.,3.E9×(3.E8+3.E8)÷(6.E3)=3×10¹⁴).

Once isolated and sequenced, binding polypeptides identified by screening the modular libraries according to the invention can be directly synthesized using conventional techniques, including solid-phase peptide synthesis, solution-phase synthesis, etc. Solid-phase synthesis is preferred. See Stewart et al., Solid-Phase Peptide Synthesis (1989), W. H. Freeman Co., San Francisco; Merrifield, J. Am. Chem. Soc., 85:2149-2154 (1963); Bodanszky and Bodanszky, The Practice of Peptide Synthesis (Springer-Verlag, New York 1984), incorporated herein by reference. Alternatively, companies providing peptide synthesis as a service (e.g., BACHEM Bioscience, Inc., King of Prussia, Pa.; Quality Controlled Biochemicals, Inc., Hopkinton, Mass.) can prepare such polypeptides commercially. Automated peptide synthesis machines, such as manufactured by Perkin-Elmer Applied Biosystems, also are available and can be employed for such syntheses.

Alternatively, binding polypeptides isolated from libraries according to the present invention also can be produced using recombinant DNA techniques, utilizing nucleic acids (polynucleotides) encoding the binding polypeptides and then expressing them recombinantly, i.e., by manipulating host cells by introduction of exogenous nucleic acid molecules in known ways to cause such host cells to produce the desired binding polypeptides. Such procedures are within the capability of those skilled in the art (see, Davis et al., Basic Methods in Molecular Biology, (1986)), incorporated by reference. Recombinant production of short peptides such as those described herein might not be practical in comparison to direct synthesis, however recombinant means of production can be very advantageous where a binding moiety is incorporated in a hybrid polypeptide or fusion protein.

Design and construction of modular display libraries in accordance with this invention will be further illustrated in the following examples. The specific parameters included in the following examples are intended to illustrate the practice of the invention, and they are not presented to in any way limit the scope of the invention.

The invention will be further described by the following non-limiting examples. The teachings of all publications cited herein are incorporated herein by reference in their entirety.

### EXEMPLIFICATION

### Example 1

A modular display library was designed having two variable regions each featuring an invariant cysteine at one amino acid position in the region. The positions of the cysteines in the display library template were separated by a span of nine amino acids, part variable and part constant region. Upon expression in phage or another genetic package, the two cysteines can form a disulfide bond, such that the display peptide forms an 11-mer cycle or loop. The template for this library has the amino acid structure: Xaa1-Xaa2-Xaa3-Cys-Xaa5-Xaa6-Xaa7-Ser-Gly-Pro-Xaa11-Xaa12-Xaa13-Cys-Xaa15-Xaa16-Xaa17 (SEQ ID NO: 4). This library is designated MTN-11/I.

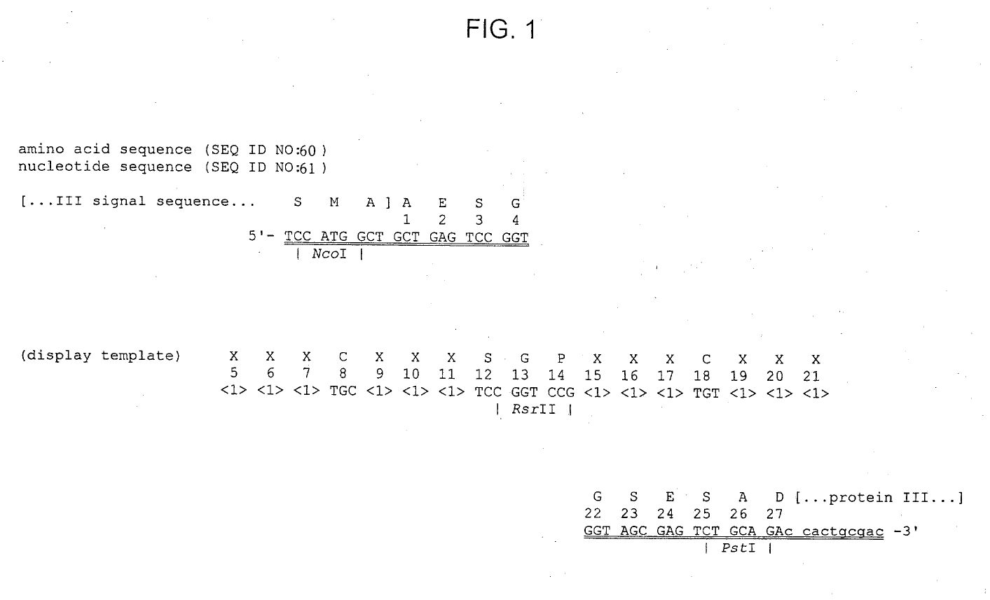

FIG. 1 shows a genetic design for MTN-11/I inserted into an M13 phage display vector, which places the display at the N-terminus of protein III. The signal peptide is cleaved before the alanine residue (amino acid no. 1) at the beginning of a four amino acid N-terminal linker for the display peptide (amino acid nos. 5-21), followed by a C-terminal linker (amino acid nos. 22-27), which is fused to the remainder of M13 protein III. The group<1>stands for a mixture of codons permitting any amino acid except cysteine. The double underscored segments can be used for PCR amplification of the 90-base synthetic oligonucleotide containing the display template. Insertion of the NcoI-PstI fragment of this oligonucleotide into a suitable display vector defines a library of 2.2×10¹⁵ possible sequences, 4.7×10⁷ for each 7-mer variable region on either side of the Ser-Gly-Pro constant region. The coding sequence for the constant region contains the recognition sequence for RsrII, 5′-CG\hat{ }G(A or T)CCG-3′ (in this case CG\hat{ }GTCCG are nucleotides 45-51 of SEQ ID NO: 18), which cleaves at the “\hat{ }” symbol, leaving non-palindromic cohesive ends (underscored).

After one or more rounds of selection, the library phage genome can be cut with RsrII and a second restriction enzyme preferably having a unique site in the genome and giving different cohesive ends (i.e., different cohesive ends from RsrII, in this example), which cuts yield two fragments, each containing one of the segments encoding a variable region of the template. Preferably the second restriction enzyme also has a non-palindromic recognition sequence and leaves cohesive ends, so that correct orientation of the segments upon religation is promoted. Suitable second restriction sites include those for BsrGI, AlwNI, NgoMIV, DraIII, BssSI, BglI, with AlwNI, DraIII, BssSI, and BglI being preferred since their use gives non-palindromic cohesive ends.

Secondary libraries extending the diversity of the original MTN-11/I library can be formed by separating the fragments, mixing the fragments in any desired proportion, religating, and transforming into cells. Alternatively, either fragment from the pooled selectants of the initial rounds of selection can be crossed back into the original library, i.e., by combining one fragment from the selectants with the opposite fragment of the original library. Alternatively, the genomes of the selectants can be cleaved, optionally separated and remixed, and recrossed with themselves. A preferred process is to digest both the RF DNA of the pool of selected phage and the RF DNA of the original (unselected) library with the same pair of restriction enzymes (i.e., unique restrictions sites corresponding to the constant (Z) region site and another unique site elsewhere in the RF DNA), mix the DNA fragments from the digestions in approximately equimolar amounts, religate, and transform to obtain a new library of, for example, ˜10⁹ transformants. This latter procedure is illustrated in the next example.

### Example 2

A modular display library was constructed having two 8-mer variable regions each featuring an invariant cysteine at one amino acid position in the region. The positions of the cysteines in the display library template were separated by a span of eleven amino acids, part variable and part constant region, such that upon expression in phage, the two cysteines would form a disulfide bond and display a 13-mer cycle or loop. The template for this library has the amino acid structure: Xaa1-Xaa2-Xaa3-Cys-Xaa5-Xaa6-Xaa7-Xaa8-Ser-Gly-Pro-Xaa12-Xaa13-Xaa14-Xaa15-Cys-Xaa17-Xaa18-Xaa19 (SEQ ID NO: 3). This library is designated MTN-13/I.

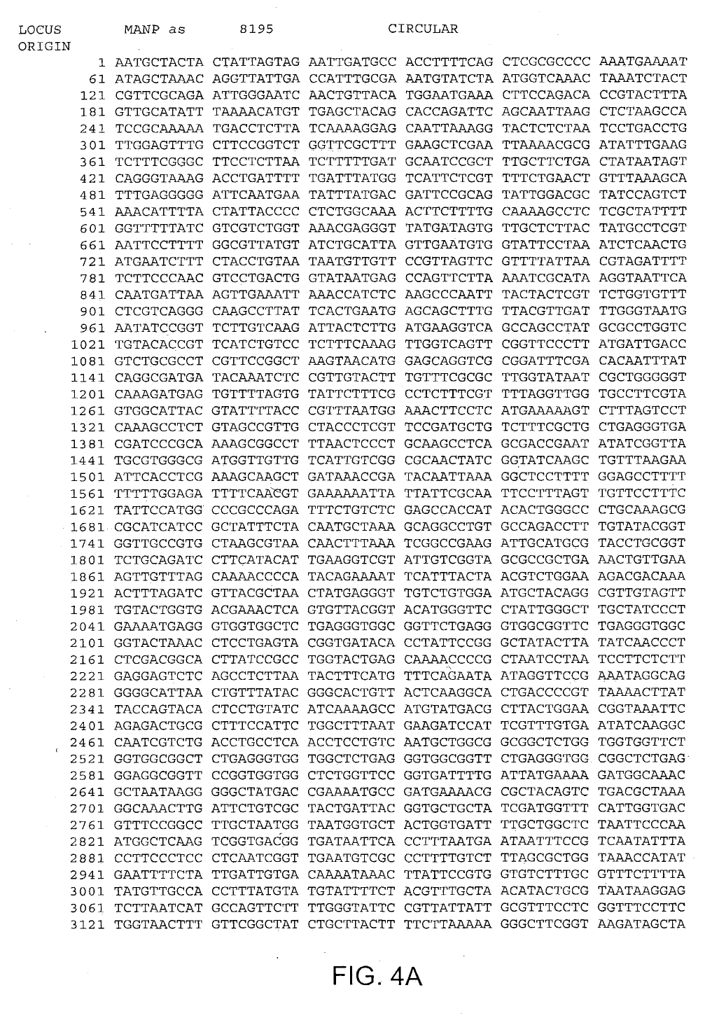

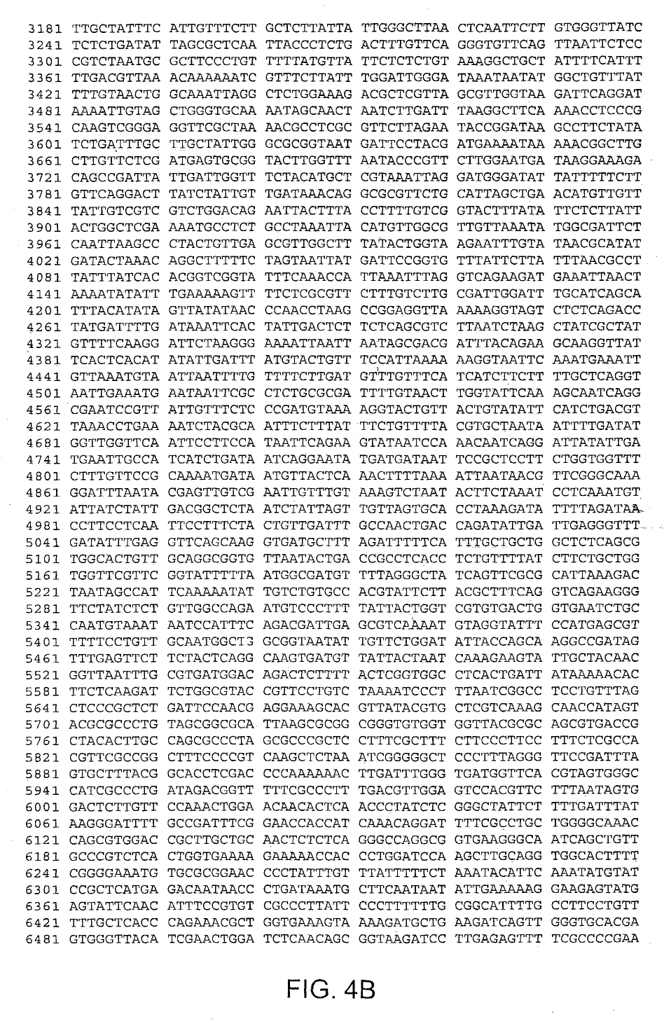

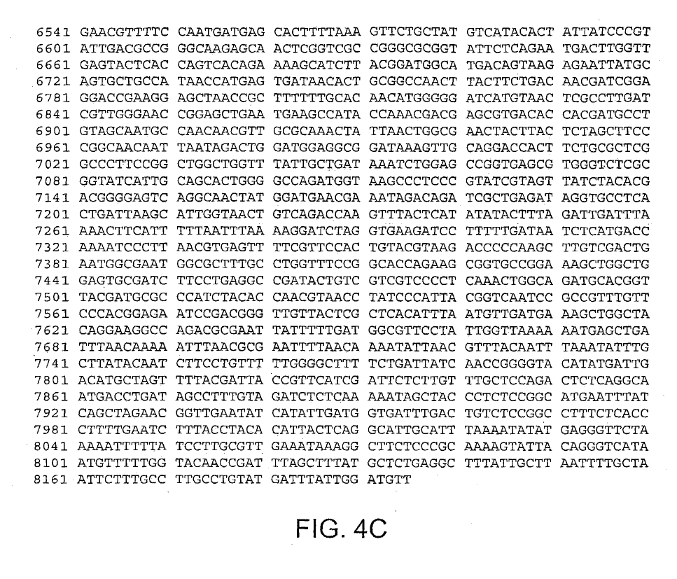

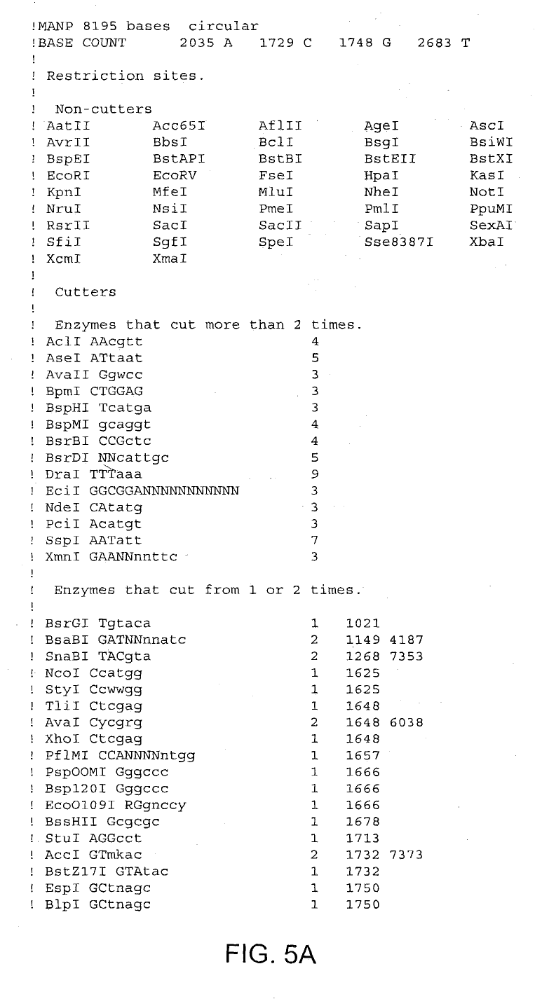

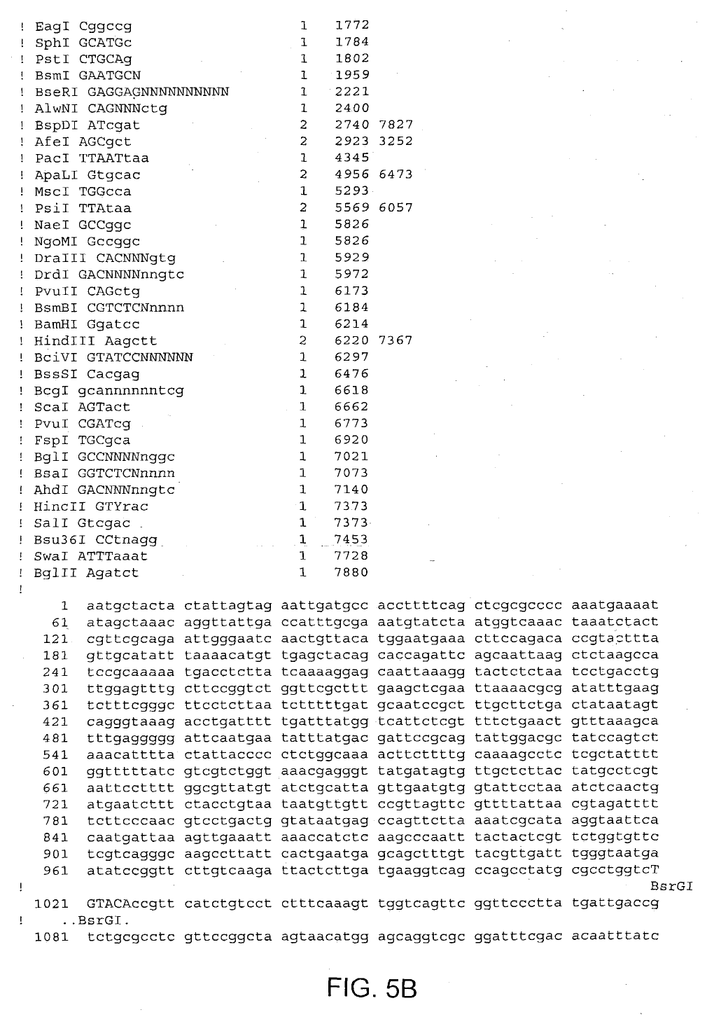

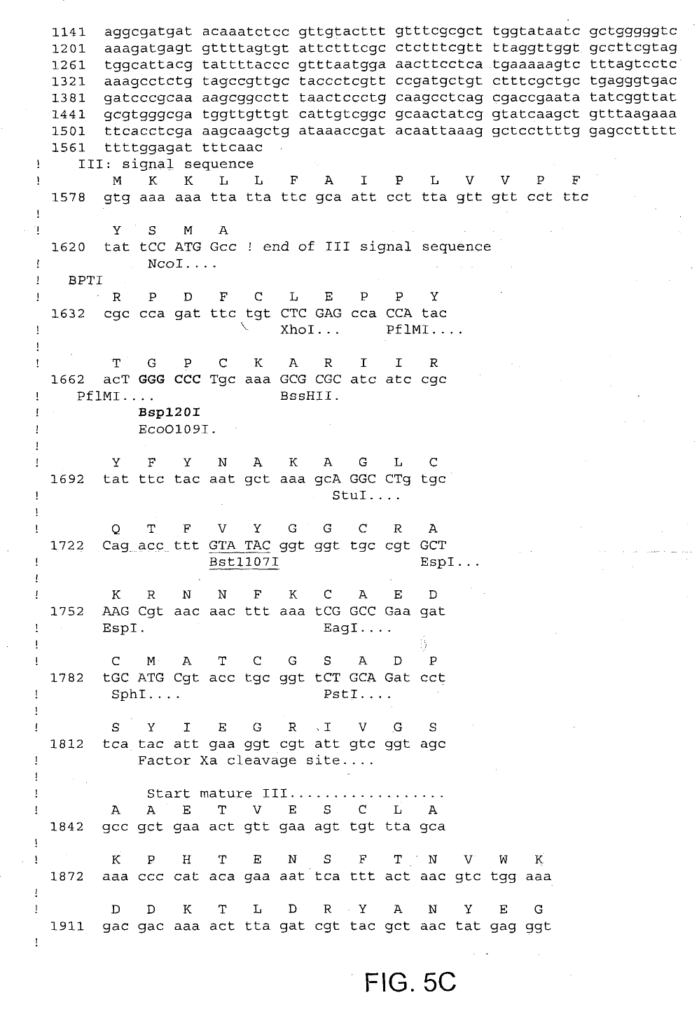

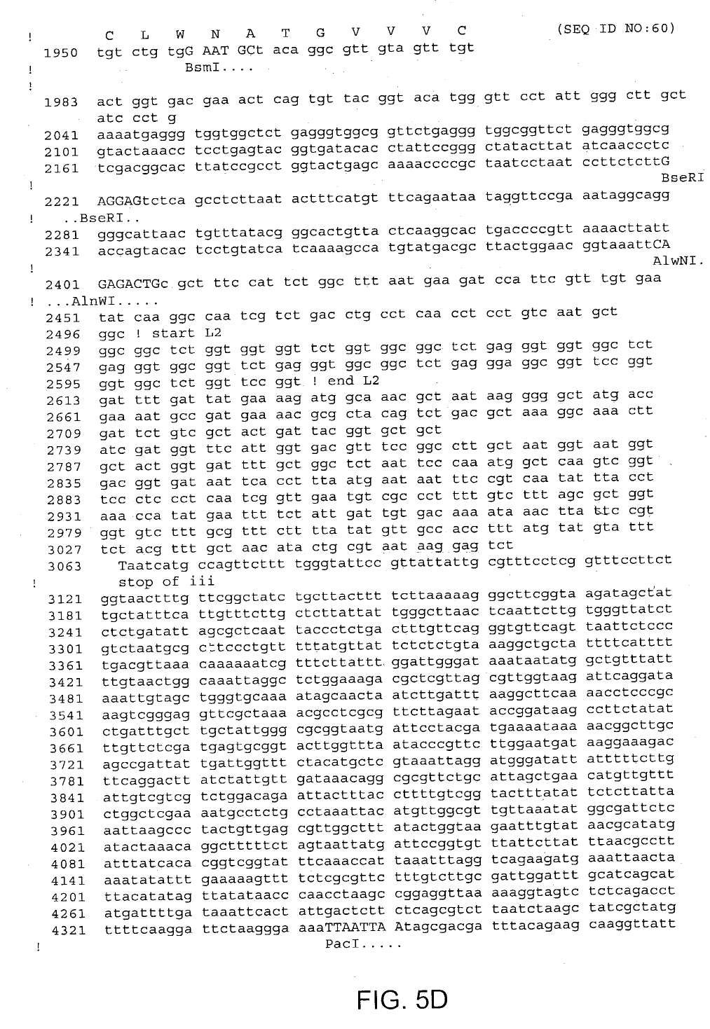

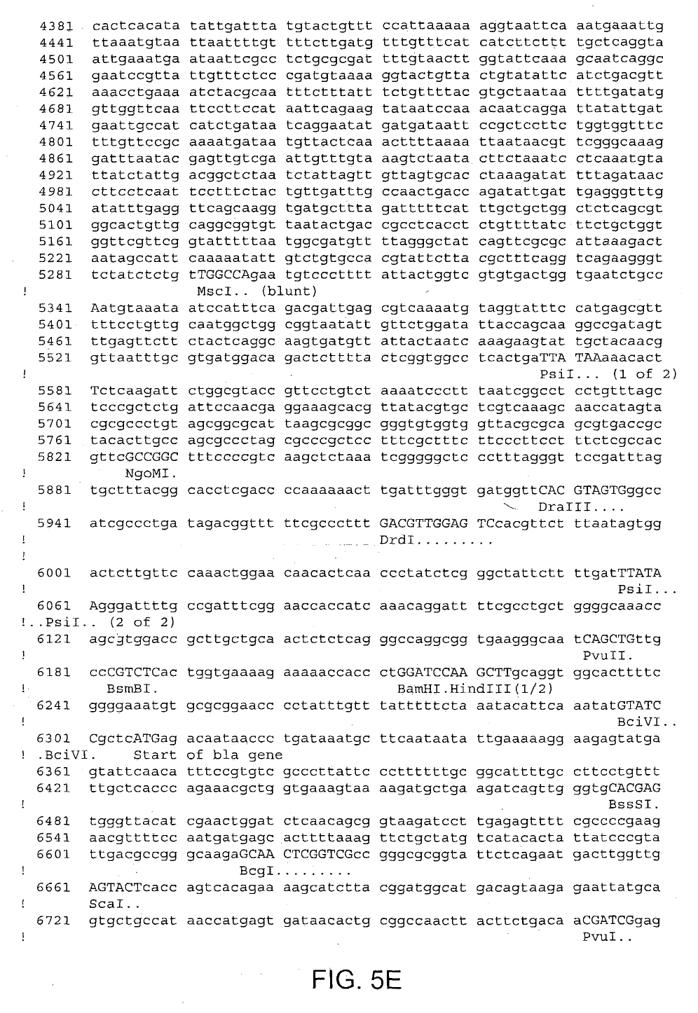

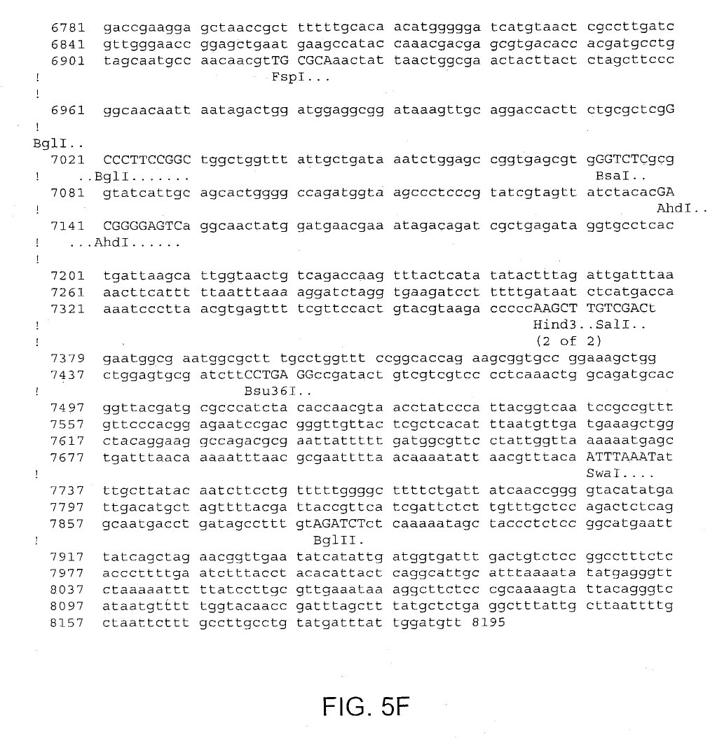

FIG. 2 shows the genetic design for MTN-13/I for insertion into an M13 phage display vector (MANP, Dyax Corp. Cambridge, Mass., see FIGS. 4A-4C), which places the display at the N-terminus of protein III. The signal peptide is cleaved before the alanine residue (amino acid no. 1) at the beginning of a four amino acid N-terminal linker for the display peptide (amino acid nos. 5-23), followed by a C-terminal linker (amino acid nos. 24-29), which is fused to the remainder of M13 protein III. The group<1>stands for codons permitting any amino acid except cysteine. Primers based on the double underscored segments in FIG. 2 were used for PCR amplification of the 96-base synthetic oligonucleotide containing the display template. Insertion of the NcoI-PstI fragment of this oligonucleotide into the similarly NcoI-PstI cut MANP vector provided vectors encoding a library of 8.0×10¹⁷ possible sequences. A phage display library of approximately 4×10⁹ transformants was obtained.

The two 8-mer variable regions of the template are connected with a constant region consisting of a tripeptide, Ser-Gly-Pro. The coding sequence for this constant region contains a recognition sequence for RsrII, 5′-CGGTCCG-3′ (nucleotides 48-54 of SEQ ID NO: 17), which leaves non-palindromic cohesive ends.

Polypeptide binders were selected against a convenient target, in this case the kinase domain region (KDR), also known as VEGF Receptor-2. To prepare a selection target, chimeric fusions of Ig Fc region with human KDR (#357-KD-050) and human Trail R4 (#633-TR-100) were purchased in carrier-free form (no BSA) from R & D Systems (Minneapolis, Minn.). Trail R4 Fc is an irrelevant Fc fusion protein with the same Fc fusion region as the target Fc fusion (KDR Fc) and was used to deplete the libraries of Fc binders. Protein A Magnetic Beads (#100.02) were purchased from Dynal. Heparin (#H-3393) was purchased from Sigma Chemical Company (St. Louis, Mo.). A 2-component tetramethyl benzidine (TMB) system was purchased from Kirkegaard and Perry (KPL, Gaithersburg, Md.).

In the selection procedure, microtiter plates were washed with a Bio-Tek 404 plate washer (Winooski, Vt.). ELISA signals were read with a Bio-Tek plate reader (Winooski, Vt.). Agitation of 96-well plates was on a LabQuake shaker (Labindustries, Berkeley, Calif.).

KDR Selection Protocol in the Presence of Heparin

Protein A Magnetic Beads (Dynal) were blocked once with 1× PBS (pH 7.5), 0.01% Tween-20, 0.1% HSA (Blocking Buffer) for 30 minutes at room temperature and then washed five times with 1× PBS (pH 7.5), 0.01% Tween-20, 5 μg/ml heparin (PBSTH Buffer).

The library was depleted against Trail R4 Fc fusion (an irrelevant Fc fusion) and then selected against KDR Fc fusion. 10¹¹ plaque forming units (pfu) from the library per 100 μl PBSTH were screened.

To prepare the KDR target beads, 500 μl of KDR-Fc fusion (0.1 =g/μl stock in PBST (no heparin)) were added to 500 μl of washed, blocked beads. The KDR-Fc fusion was allowed to bind overnight with agitation at 4° C. The next day, the beads were washed 5 times with PBSTH.

To prepare the irrelevant Fc fusion beads, 500 μl of Trail R4-Fc fusion (0.1 μg/μl stock in PBST (no heparin)) were added to 1000 μl of washed, blocked Protein A magnetic beads. The fusion was allowed to bind to the beads overnight with agitation at 4° C. The next day, the magnetic beads were washed 5 times with PBSTH.

The phage library was incubated with 50 μl of Trail R4 Fc fusion beads on a Labquake shaker for 1 hour at room temperature (RT). After incubation, the phage supernatant was removed and incubated with another 50 μl of Trail R4 beads. This was repeated for a total of 5 rounds of depletion, to remove non-specific Fc fusion and bead binding phage from the library. The depleted library was added to 100 μl of KDR-Fc beads and allowed to incubate on a LabQuake shaker for 1 hour at RT. Beads were then washed as rapidly as possible with 5×1 ml PBSTH using a magnetic stand (Promega) to separate the beads from the wash buffer. Phage still bound to beads after the washing were competition-eluted using soluble VEGF₁₆₅ (#100-20) purchased in carrier-free form from Peprotech (Rocky Hill, N.J.) as follows: The beads were incubated with 250 μl of VEGF (50 μg/ml,˜1 μM) overnight at room temperature (RT) on a LabQuake shaker. The beads after VEGF elution were mixed with cells to amplify the phage still bound to the beads, i.e., KDR-binding phage that had not been competed off by the VEGF incubation. After approximately 15 minutes at room temperature, the phage/cell mixture was spread onto a Bio-Assay Dish (243×243×18 mm, Nalge Nunc) containing 250 ml of NZCYM agar with 50 μg/ml of ampicillin. The plate was incubated overnight at 37° C. The next day, each amplified phage culture was harvested from its respective plate. Over the next day, the input, output and amplified phage cultures were titered for FOI (i.e., Fraction of Input=phage output divided by phage input).

This selection procedure was repeated in the absence of heparin in all binding buffers, i.e., substituting PBST (PBS (pH 7.5), 0.01% Tween-20) for PBSTH in all steps.

KDR Screening Assay

100 μl of KDR-Fc fusion or Trail R4-Fc fusion (1 μg/ml) were added to duplicate Immulon II plates, to every well, and allowed to incubate at 4° C. overnight. Each plate was washed twice with PBST (PBS, 0.05% Tween-20). The wells were filled to the top with 1× PBS, 1% BSA and allowed to incubate at RT for 2 hours. Each plate was washed once with PBST (PBS, 0.05% Tween-20).

Once the plates were prepared, each overnight phage culture was diluted 1:1 (or to 10¹⁰ pfu if using purified phage stock) with PBS, 0.05% Tween-20, 1% BSA. 100 μl of each diluted culture was added and allowed to incubate at RT for 2-3 hours. Each plate was washed 5 times with PBST. The binding phage were visualized by adding 100 μl of a 1:10,000 dilution of HRP-anti-M13 antibody conjugate (Pharmacia), diluted in PBST, to each well, then incubating at room temperature for 1 hr. Each plate was washed 7 times with PBST (PBS, 0.05% Tween-20), then the plates were developed with HRP substrate (˜10 minutes) and the absorbance signal (630 nm) detected with a plate reader.

KDR binding phage from four rounds of screening were recovered and amplified, and standard DNA sequencing methods were used to determine the sequences of the display peptides responsible for the binding. The binding peptides of the phage isolates recovered are set forth in Table 1, below.

Table 1

Initial MTN-13/I KDR Binding Peptides Selected

| | | |
|---|---|---|
| SEQ ID NO: | Sequence | frequency |

| | | |
|---|---|---|
| 5 | RLDCDKVFSGPHGKICVNY | 16 |
| 6 | WQECTKVLSGPGQFLCSYG | 3 |
| 7 | SNKCDHYQSGPYGAVCLHY | 2 |
| 8 | SPHCQYKISGPFGPVCVNY | 2 |
| 9 | WYRCDFNMSGPDFTECLYP | 2 |
| 10 | RLDCDMVFSGPHGKICVNY | 1 |
| 11 | KRCDTTHSGPHGIVCVVY | 1 |
| 12 | AHQCHHWTSGPYGEVCFNY | 1 |
| 13 | YDKCSSRFSGPFGEICVNY | 1 |
| 14 | MGGCDFSFSGPFGQICGRY | 1 |
| 15 | RTTCHHQISGPFGDVCVSY | 1 |
| 16 | WMQCNMSASGPKDMYCEYD | 1 |
| 17 | GISCKWIWSGPDRWKCHHF | 1 |
| 18 | WQVCKPYVSGPAAFSCKYE | 1 |
| 19 | GWWCYRNDSGPKPFHCRIK | 1 |
| 20 | EGWCWFIDSGPWKTWCEKQ | 1 |
| 21 | FPKCKFDFSGPPWYQCNTK | 1 |

DNA of the phage isolates from the first four rounds of selection against KDR target (Table 1) was isolated and cleaved with RsrII and BglI (giving 5′-GTC and TTC-3′ cohesive ends), both unique restriction sites in the MANP vector. This cleavage yielded two DNA segments of 2.8 kb and 5.3 kb. The cleaved DNA was mixed with an approximately equimolar amount of similarly cleaved phage vector DNA of the original MTN-13/I unselected library. These DNA molecules were ligated and used to transform cells. A secondary phage display library of 4×10⁹ transformants was obtained.

The secondary library was screened for KDR binding polypeptides as before, and the isolates collected and sequenced. The binding polypeptides of these isolates are shown in Table 2, below.

Table 2

Isolates of Secondary MTN-13/I Library (Round 1)

| | | |
|---|---|---|
| SEQ ID NO: | Sequence | frequency |

| | | |
|---|---|---|
| 5 | RLDCDKVFSGPHGKICVNY | 28 |
| 22 | RTTCHHQISGPHGKICVNY | 6 |
| 23 | SNKCDHYQSGPHGKICVNY | 3 |
| 24 | WQECTKVLSGPGQFECEYM | 2 |
| 25 | RLDCDKVFSGPYGRVCVKY | 2 |
| 26 | WQECTKVLSGPGQFSCVYG | 1 |
| 27 | RLDCDKVFSGPYGNVCVNY | 1 |
| 28 | RLDCDKVFSGPSMGTCKLQ | 1 |
| 29 | RTTCHHHISGPHGKICVNY | 1 |
| 30 | QFGCEHIMSGPHGKICVNY | 1 |
| 31 | PVHCSHTISGPHGKICVNY | 1 |
| 32 | SVTCHFQMSGPHGKICVNY | 1 |
| 33 | PRGCQHMISGPHGKICVNY | 1 |
| 34 | RTTCHHQISGPHGQICVNY | 1 |
| 35 | WTICHMELSGPHGKICVNY | 1 |
| 36 | FITCALWLSGPHGKICVNY | 1 |
| 37 | MGGCDFSFSGPHGKICVNY | 1 |
| 38 | KDWCHTTFSGPHGKICVNY | 1 |
| 39 | AWGCDNMMSGPHGKICVNY | 1 |
| 40 | SNKCDHIMSGPHGKICVNY | 1 |
| 41 | SNKCDHYQSGPFGDICVMY | 1 |
| 42 | SNKCDHYQSGPFGDVCVSY | 1 |
| 43 | SNKCDHYQSGPFGDICVSY | 1 |
| 44 | RTTCHHQISGPFGPVCVNY | 1 |
| 45 | RTTCHHQISGPYGDICVKY | 1 |
| 46 | RYKCPRDLSGPPYGPCSPQ | 1 |

A second round of selection against a KDR target was performed on the secondary library, and the polypeptides isolated are set forth in Table 3, below.

Table 3

Isolates of Secondary MTN-13/I Library (Round 2)

| SEQ ID NO: | Sequence | frequency |
|---|---|---|
| 24 | WQECTKVLSGPGQFECEYM | 25 |
| 23 | SNKCDHYQSGPHGKICVNY | 8 |
| 5 | RLDCDKVFSGPHGKICVNY | 6 |
| 26 | WQECTKVLSGPGQFSCVYG | 3 |
| 47 | WQECTKVLSGPGTFECSYE | 2 |
| 22 | RTTCHHQISGPHGKICVNY | 2 |
| 48 | SNKCDHYQSGPYGEVCFNY | 1 |
| 49 | RLDCDKVFSGPYGKVCVSY | 1 |
| 50 | RLDCDKVFSGPDTS-CGSQ | 1 |
| 51 | RLDCDKVFSGPHGKICVRY | 1 |
| 52 | RVDCDKVISGPHGKICVNY | 1 |
| 53 | EFHCHHIMSGPHGKICVNY | 1 |
| 54 | HNRCDFKMSGPHGKICVNY | 1 |
| 55 | WQECTKVLSGPNSFECKYD | 1 |
| 56 | WDRCERQISGPGQFSCVYG | 1 |

From the above results, SEQ ID NO: 5 could have been over-represented in the unselected recombined library. The prevalence of SEQ ID NO: 5 decreases in subsequent rounds of selection, indicating that the recombined variable region components have better binding characteristics. The reappearance of particular right or left variable regions in the original and secondary libraries (see, e.g., right variable region in SEQ ID NOS: 23, 5, 22 and 52-54; see, e.g., left variable region in SEQ ID NOS: 7, 23, 41-43 and 48) indicates favored binding moieties.

Analysis of the isolated sequences shows that the sequences in Table 2 have either a first variable region (N-terminal to the constant region) or a second variable region (C-terminal to the constant region) found in Table 1, except for SEQ ID NOS: 41-43 and 46. Of the sequences in Table 3, all have at least one variable region from Table 1, except SEQ ID NO: 56. SEQ ID NOS: 5, 22-24, 48 and 55 all exhibit two variable regions from Table 1; SEQ ID NOS: 26, 47 and 49-51 have a first variable region from Table 1; and SEQ ID NOS: 52-54 have the second variable region from SEQ ID NO: 5 of Table 1.

The first variable region of SEQ ID NO: 5, which is the most frequently isolated sequence in Table 1, occurs in SEQ ID NOS: 25, 27, 42 and 49-51; the second half of SEQ ID NO: 6 occurs SEQ ID NOS: 10, 22, 23, 29-33, 35-40 and 52-54. The first and second variable regions of other polypeptides in Table 1 can be traced into Tables 2 and 3.

### Example 3

Binding affinity of selected peptides from the various rounds of selection was tested using a BIAcore surface plasmon resonance spectrophotometer. Polypeptides selected from Tables 1, 2, and 3 were synthesized by solid phase synthesis using the sequence as shown in the tables above and an N-terminal flanking peptide, acetyl-Ser-Gly-, and a C-terminal flanking peptide, -Gly-Ser. A KDR-Fc fusion protein target was immobilized on a BIAcore chip, and the synthetic polypeptides were flowed over the chip to measure the K_{D} of the polypeptides with respect to the KDR target. Polypeptides corresponding to SEQ ID NOS: 6, 7, 23 and 24 showed no binding; polypeptides corresponding to SEQ ID NOS: 5 and 12 showed weak binding, with a K_{D} >2 μM; a polypeptide corresponding to SEQ ID NO: 49 showed a K_{D}=1 μM; a polypeptide corresponding to SEQ ID NO: 22 showed a K_{D}=0.65 μM.

SEQ ID NO: 49, which exhibits 1 micromolar binding to KDR target as a free polypeptide, contains the first variable region of SEQ ID NO: 5 (Table 1) and a second variable region not seen in Table 1. It is probable that the combination of variable regions in SEQ ID NO: 49 resulted from recombination of a selected first variable region with a second variable region from the original library. SEQ ID NO: 22, the highest affinity binder, exhibiting a K_{D} of 650 nanomolar with respect to the KDR target, comprises the first variable region of SEQ ID NO: 15 (Table 1) and the second variable region seen in both SEQ ID NO: 5 and SEQ ID NO: 10 (Table 1). Thus, recombination of the first and second variable region sequences yielded higher affinity binders than were isolated from the unrecombined library.

### Example 4

A modular linear display library was designed having two variable regions of eight amino acids joined by a constant region tripeptide. The template for this library has the amino acid structure: Xaa1-Xaa2-Xaa3-Xaa4-Xaa5-Xaa6-Xaa7-Xaa8-Pro-Ser-Gly-Xaa12-Xaa13-Xaa14Xaa15-Xaa16-Xaa17-Xaa18-Xaa19.

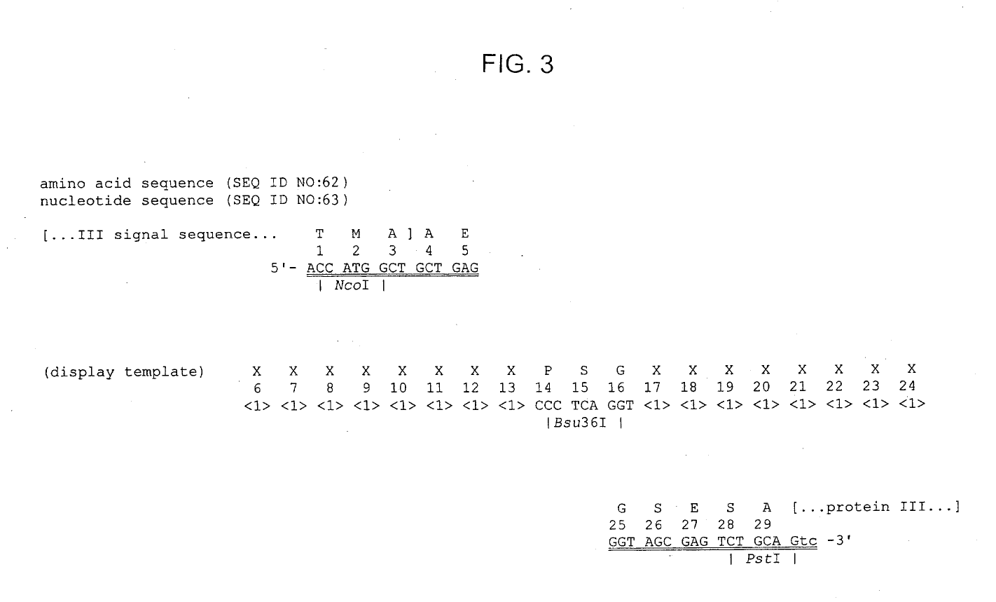

FIG. 3 shows a genetic design for the modular linear display library for insertion into an M13 phage display vector, which places the display at the N-terminus of protein III. The signal peptide is expected to cleave before the alanine residue (amino acid no. 4) in an N-terminal linker for the display peptide (amino acid nos. 6-24), followed by a C-terminal linker (amino acid nos. 25-29), which is fused to the remainder of M13 protein III. The group<1>stands for codons permitting any amino acid except cysteine. The double underscored segments can be used for PCR amplification of the 90-base synthetic oligonucleotide containing the display template. Insertion of the NcoI-PstI fragment of this oligonucleotide into a suitable display vector defines a library of 2.9×10²⁰ possible sequences, 1.7×10¹⁰ for each 8-mer variable region on either side of the Ser-Gly-Pro constant region. As shown in FIG. 3, the coding sequence for the constant region contains the recognition sequence for Bsu36I, 5′-CCTCAGG-3′, which cleaves to leave cohesive ends.

After one or more rounds of selection, the library phage genome can be cut with Bsu36I and a second restriction enzyme preferably having a unique site in the genome, giving two fragments, each containing one of the segments encoding a variable region of the template. Preferably the second restriction enzyme also has a non-palindromic recognition sequence and leaves cohesive ends, so that correct orientation of the segments upon religation is promoted.

Secondary libraries extending the diversity of the original modular linear library can be formed by separating the fragments, mixing the fragments in any desired proportion, religating, and transforming into cells. Alternatively, either fragment from the pooled selectants of the initial rounds of selection can be crossed back into the original library, i.e., by combining one fragment from the selectants with the opposite fragment of the original library. Alternatively, the genomes of the selectants can be cleaved, optionally separated and remixed, and recrossed with themselves.

While this invention has been particularly shown and described with references to preferred embodiments thereof, it will be understood by those skilled in the art that various changes in form and details can be made therein without departing from the scope of the invention encompassed by the appended claims.

What is claimed is:

1. A library of display vectors comprising a plurality of DNA molecules comprising a general structure, R1-Z-R2, wherein R1 and R2 are independently variable regions, and wherein Z is a constant region that includes a cleavage site for a restriction endonuclease.

2. The library of claim 1, wherein R1 and R2 are at least 15 bases, and wherein at least 12 bases are variable.

3. The library of claim 1, wherein R1 and R2 are between about 6 and about 36 bases in length.

4. The library of claim 3, wherein Z is between about 6 and about 9 bases in length.

5. The library of claim 1, wherein R1 and R2 are the same length.

6. The library of claim 1, wherein R1 and R2 are different lengths.

7. The library of claim 1, wherein R1 and or R2 comprise positions that are held constant.

8. The library of claim 1, wherein the R1-Z-R2 region is flanked by a restriction endonuclease cleavage site on both sides.

9. The library of claim 1, wherein R1 and R2 each encode a peptide of 7 amino acids having the formula: Xaa1-Xaa2-Xaa3-Cys-Xaa5-Xaa6-Xaa7.

10. The library of claim 9, wherein each Xaa can be any amino acid except cysteine, and wherein Z encodes a tripeptide Ser-Gly-Pro.

11. The library of claim 1, wherein R1 comprises the sequence 5′-NNK NNK NNK TGY NNK NNK NNK-3′, and wherein R2 comprises the sequence 5′-NNK NNK NNK TGY NNK NNK NNK-3′.

12. The method of claim 11, wherein NNK represents a collection of codons wherein each amino acid is encoded by one codon of the collection.

13. The method of claim 11, wherein NNK represents a collection of codons wherein each amino acid except cysteine is encoded by one codon of the collection.

14. The library of claim 1, wherein the DNA molecules comprise the sequence: 5′-NNK NNK NNK TGY NNK NNK NNK TCC GGT CCG NNK NNK NNK TGY NNK NNK NNK-3′.

15. The method of claim 14, wherein NNK represents a collection of codons wherein each amino acid is encoded by one codon of the collection.

16. The method of claim 14, wherein NNK represents a collection of codons wherein each amino acid except cysteine is encoded by one codon of the collection.

17. The library of claim 1, wherein R1 encodes a peptide having the sequence:
Xaa1-Xaa2-Xaa3-Cys-Xaa5-Xaa6-Xaa7-Xaa8 wherein each Xaa can be any amino acid except cysteine, and wherein R2 encodes a peptide having the sequence: Xaa1-Xaa2-Xaa3-Xaa4-Cys-Xaa6-Xaa7-Xaa8 wherein each Xaa can be any amino acid except cysteine, and wherein Z encodes the tripeptide, Ser-Gly-Pro.

18. The library of claim 1, wherein the restriction endonuclease site in Z is selected from the group consisting of: AccI, AflIII, AlwNI, AvaI, BanI, BanII, BbsI, BbvCI, BglI, BlpI, BsaI, BsaJI, BseYI, BsiEI, BsiHKAI, Bsp1286I, BsmI, BsmBI, BsrI, BsrI, BsrDI, BsrDI, BssSI, BssSI, BstEII, Bsu36I, DraIII, DsaI, EcoO109I, EspI, PpuMI, RsrII, SexAI, SfcI, StyI.

19. The library of claim 18, wherein the restriction site in Z is unique in the vector.

20. The library of claim 18, wherein the restriction site in Z occurs at one other site in the vector.

21. A library of circular DNA display vectors comprising a display coding sequence comprising the general structure R1-Z-R2, wherein R1 and R2 are independently variable regions each of at least 15 bases wherein at least 12 bases are variable, and wherein Z is a constant region that includes a cleavage site of a first restriction endonuclease, and a second endonuclease site separated from R1-Z-R2 by at least 10 bases.

22. The library of claim 21, wherein R1 and R2 are at least 15 bases, and wherein at least 12 bases are variable.

23. The library of claim 22, wherein the first and second endonucleases produce different cohesive ends.

24. The library of claim 22, wherein the first endonuclease produces cohesive ends that are not palindromic.

25. The library of claim 22, wherein the second endonuclease produces cohesive ends that are not palindromic.

26. The library of claim 22, wherein the second endonuclease is a type IIS endonuclease.

27. The library of claim 22, wherein the restriction enzymes that recognize said first and second restriction sites are different.

28. The library of claim 22, wherein the restriction enzymes that recognize said first and second restriction sites are the same.

29. The library of claim 21, wherein the restriction endonuclease site in Z is selected from the group consisting of: AccI, AflIII, AlwNI, AvaI, BanI, BanII, BbsI, BbvCI, BglI, BlpI, BsaI, BsaJI, BseYI, BsiEI, BsiHKAI, Bsp1286I, BsmI, BsmBI, BsrI, BsrI, BsrDI, BsrDI, BssSI, BssSI, BstEII, Bsu36I, DraIII, DsaI, EcoO109I, EspI, PpuMI, RsrII, SexAI, SfcI, StyI.

30. The library of claim 21, wherein the restriction site in Z is unique in the vector.

31. The library of claim 21, wherein the restriction site in Z occurs at one other site in the vector.

32. A method for obtaining a binding peptide comprising the steps of:
selecting for phage in a library according to claim 1, wherein a displayed peptide binds to a target of interest;
obtaining RF DNA for the selected phage;
cleaving the library RF DNA at the first and second restriction sites;
cleaving the library RF DNA at the first and second restriction sites;
mixing the selected RF DNA fragments and the library RF DNA fragments;
ligating the mixed fragments;
introducing the ligated fragments into cells, such that phage displaying a new library are produced; and
selecting and sequencing binding phage from the new library, thereby obtaining the binding peptide.

33. A method for producing a modular phage display library comprising:
a) preparing a multiplicity of DNA molecules comprising a DNA sequence of the formula R1 -Z-R2, wherein R1 and R2 are, independently, variable regions of at least 15 bases (5 codons) wherein at least 12 bases are variable, and wherein Z is a constant region of at least 6 bases that includes the cleavage site of a restriction endonuclease,
b) inserting sthe DNA molecules into one or more phage display vector cassettes (e.g., such as MANP, FIGS. 4A-4C), and
c) transfecting host bacteria with the phage display vector cassettes, thereby creating the modular phage display library.

34. The method of claim 32, wherein R1 and R2 are at least 15 bases, and wherein at least 12 bases are variable.

35. The method of claim 32, wherein R1 and R2 are between about 6 and about 36 bases in length.

36. The method of claim 33, wherein R1 and R2 are between about 6 and about 9 bases in length.

37. The method of claim 32, wherein R1 and R2 are the same length.

38. The method of claim 32, wherein R1 and R2 are different lengths.

39. The method of claim 32, wherein R1 and or R2 comprise positions that are constant.

40. The method of claim 32, wherein the R1-Z-R2 region is flanked by a restriction endonuclease cleavage site on both sides.

41. The method of claim 32, wherein R1 and R2 each encode a peptide of 7 amino acids having the formula: Xaa1-Xaa2-Xaa3-Cys-Xaa5-Xaa6-Xaa7.

42. The method of claim 33, wherein each Xaa can be any amino acid except cysteine, and wherein Z encodes a tripeptide Ser-Gly-Pro.

43. The method of claim 32, wherein R1 comprises the sequence 5′-NNK NNK NNK TGY NNK NNK NNK-3′, and wherein R2 comprises the sequence 5′-NNK NNK NNK TGY NNK NNK NNK-3′.

44. The method of claim 32, wherein NNK represents a collection of codons wherein each amino acid is encoded by one codon of the collection.

45. The method of claim 32, wherein NNK represents a collection of codons wherein each amino acid except cysteine is encoded by one codon of the collection.

46. The method of claim 32, wherein the DNA molecules comprise the sequence: 5′-NNK NNK NNK TGY NNK NNK NNK TCC GGT CCG NNK NNK NNK TGY NNK NNK NNK-3′.

47. The method of claim 46, wherein NNK represents a collection of codons wherein each amino acid is encoded by one codon of the collection.

48. The method of claim 46, wherein NNK represents a collection of codons wherein each amino acid except cysteine is encoded by one codon of the collection.

49. The method of claim 32, wherein R1 encodes a peptide having the sequence:
Xaa1-Xaa2-Xaa3-Cys-Xaa5-Xaa6-Xaa7-Xaa8 wherein each Xaa can be any amino acid except cysteine, and wherein R2 encodes a peptide having the sequence: Xaa1-Xaa2-Xaa3-Xaa4-Cys-Xaa6-Xaa7-Xaa8 wherein each Xaa can be any amino acid except cysteine, and wherein Z encodes the tripeptide, Ser-Gly-Pro.

50. The method of claim 32, wherein the restriction endonuclease site in Z is selected from the group consisting of: AccI, AflIII, AlwNI, AvaI, BanI, BanII, BbsI, BbvCI, BglI, BlpI, BsaI, BsaJI, BseYI, BsiEI, BsiHKAI, Bsp1286I, BsmI, BsmBI, BsrI, BsrI, BsrDI, BsrDI, BssSI, BssSI, BstEII, Bsu36I, DraIII, DsaI, EcoO109I, EspI, PpuMI, RsrII, SexAI, SfcI, StyI.

51. The library of claim 32, wherein the restriction site in Z is unique in the vector.

52. The library of claim 32, wherein the restriction site in Z occurs at one other site in the vector.

53. A method for producing a recombinatorial phage display library, comprising:
a) digesting phage display vector cassettes from the modular display library with a restriction endonuclease that cleaves within the constant region Z and a second restriction endonuclease that cleaves the vector cassette so as to yield a first and a second vector fragment, said first vector fragment including R1 and said second vector fragment including R2;
b) mixing the vector fragments together and religating the fragments to form recombinatorial phage vector cassettes; and
c) transfecting host bacteria with the recombinatorial phage vector cassettes,
thereby producing the recombinatorial phage display library.

54. The method of claim 53, wherein R1 and R2 are at least 15 bases, and wherein at least 12 bases are variable.

55. The method of claim 53, wherein R1 and R2 are between about 6 and about 36 bases in length.

56. The method of claim 53, wherein R1 and R2 are between about 6 and about 9 bases in length.

57. The method of claim 53, wherein R1 and R2 are the same length.

58. The method of claim 53, wherein R1 and R2 are different lengths.

59. The method of claim 53, wherein R1 and or R2 comprise positions that are held constant.

60. The method of claim 53, wherein the R1-Z-R2 region is flanked by a restriction endonuclease cleavage site on both sides.

61. The method of claim 53, wherein R1 and R2 each encode a peptide of 7 amino acids having the formula: Xaa1-Xaa2-Xaa3-Cys-Xaa5-Xaa6-Xaa7.

62. The method of claim 61, wherein each Xaa can be any amino acid except cysteine, and wherein Z encodes a tripeptide Ser-Gly-Pro.

63. The method of claim 53, wherein R1 comprises the sequence 5′-NNK NNK NNK TGY NNK NNK NNK-3′, and wherein R2 comprises the sequence 5′-NNK NNK NNK TGY NNK NNK NNK-3′.

64. The method of claim 63, wherein NNK represents a collection of codons wherein each amino acid is encoded by one codon of the collection.

65. The method of claim 64, wherein NNK represents a collection of codons wherein each amino acid except cysteine is encoded by one codon of the collection.

66. The method of claim 53, wherein the DNA molecules comprise the sequence: 5′-NNK NNK NNK TGY NNK NNK NNK TCC GGT CCG NNK NNK NNK TGY NNK NNK NNK-3′.

67. The method of claim 66, wherein NNK represents a collection of codons wherein each amino acid is encoded by one codon of the collection.

68. The method of claim 67, wherein NNK represents a collection of codons wherein each amino acid except cysteine is encoded by one codon of the collection.

69. The method of claim 53, wherein R1 encodes a peptide having the sequence:
Xaa1-Xaa2-Xaa3-Cys-Xaa5-Xaa6-Xaa7-Xaa8 wherein each Xaa can be any amino acid except cysteine, and wherein R2 encodes a peptide having the sequence: Xaa1-Xaa2-Xaa3-Xaa4-Cys-Xaa6-Xaa7-Xaa8 wherein each Xaa can be any amino acid except cysteine, and wherein Z encodes the tripeptide, Ser-Gly-Pro.

70. The method of claim 53, wherein R1 and/or R2 are isolated by a prior selection.

71. The method of claim 53, wherein the restriction endonuclease site in Z is selected from the group consisting of: AccI, AflIII, AlwNI, AvaI, BanI, BanII, BbsI, BbvCI, BglI, BlpI, BsaI, BsaJI, BseYI, BsiEI, BsiHKAI, Bsp 1286I, BsmI, BsmBI, BsrI, BsrI, BsrDI, BsrDI, BssSI, BssSI, BstEII, Bsu36I, DraIII, DsaI, EcoO109I, EspI, PpuMI, RsrII, SexAI, SfcI, StyI.

72. The library of claim 53, wherein the restriction site in Z is unique in the vector.

73. The library of claim 53, wherein the restriction site in Z occurs at one other site in the vector.


SEQUENCE LISTING (65 sequences)

SEQ ID NO: 1 - DNA, 51 bp, Artificial Sequence
  Template vector sequence
  misc_feature 1, 2, 4, 5, 7, 8, 13, 14, 16, 17, 19, 20, 31, 32, 34, 35,37, 38, 43, 44, 46, 47, 49, 50: n = any nucleotide
  misc_feature 3, 6, 9, 15, 18, 21, 33, 36, 39, 45, 48, 51: k = g or t
nnknnknnktgynnknnknnktccggtccgnnknnknnktgynnknnknnk

SEQ ID NO: 2 - DNA, 57 bp, Artificial Sequence
  Template vector sequence
  misc_feature 1, 2, 4, 5, 7, 8, 13, 14, 16, 17, 19, 20, 22, 23, 34,35,37, 38, 40, 41, 43, 44, 49, 50, 52, 53, 55, 56: n = any nucleotide
  misc_feature 3, 6, 9, 15, 18, 21, 24, 36, 39, 42, 45, 51, 54, 57: k = g or t
nnknnknnktgynnknnknnknnktccggtccgnnknnknnknnktgynnknnknnk

SEQ ID NO: 3 - protein, 19 aa, Artificial Sequence
  Template mino acid sequence
  VARIANT 1, 2, 3, 5, 6, 7, 8, 12, 13, 14, 15, 17, 18, 19: Xaa = Any Amino Acid
XXXCXXXXSGPXXXXCXXX

SEQ ID NO: 4 - protein, 17 aa, Artificial Sequence
  Template amino acid sequence
  VARIANT 1, 2, 3, 5, 6, 7, 11, 12, 13, 15, 16, 17: Xaa = Any Amino Acid
XXXCXXXSGPXXXCXXX

SEQ ID NO: 5 - protein, 19 aa, Artificial Sequence
  Selected library sequence
RLDCDKVFSGPHGKICVNY

SEQ ID NO: 6 - protein, 19 aa, Artificial Sequence
  Selected library sequence
WQECTKVLSGPGQFLCSYG

SEQ ID NO: 7 - protein, 19 aa, Artificial Sequence
  Selected library sequence
SNKCDHYQSGPYGAVCLHY

SEQ ID NO: 8 - protein, 19 aa, Artificial Sequence
  Selected library sequence
SPHCQYKISGPFGPVCVNY

SEQ ID NO: 9 - protein, 19 aa, Artificial Sequence
  Selected library sequence
WYRCDFNMSGPDFTECLYP

SEQ ID NO: 10 - protein, 19 aa, Artificial Sequence
  Selected library sequence
RLDCDMVFSGPHGKICVNY

SEQ ID NO: 11 - protein, 18 aa, Artificial Sequence
  Selected library sequence
KRCDTTHSGPHGIVCVVY

SEQ ID NO: 12 - protein, 19 aa, Artificial Sequence
  Selected library sequence
AHQCHHWTSGPYGEVCFNY

SEQ ID NO: 13 - protein, 19 aa, Artificial Sequence
  Selected library sequence
YDKCSSRFSGPFGEICVNY

SEQ ID NO: 14 - protein, 19 aa, Artificial Sequence
  Selected library sequence
MGGCDFSFSGPFGQICGRY

SEQ ID NO: 15 - protein, 19 aa, Artificial Sequence
  Selected library sequence
RTTCHHQISGPFGDVCVSY

SEQ ID NO: 16 - protein, 19 aa, Artificial Sequence
  Selected library sequence
WMQCNMSASGPKDMYCEYD

SEQ ID NO: 17 - protein, 19 aa, Artificial Sequence
  Selected library sequence
GISCKWIWSGPDRWKCHHF

SEQ ID NO: 18 - protein, 19 aa, Artificial Sequence
  Selected library sequence
WQVCKPYVSGPAAFSCKYE

SEQ ID NO: 19 - protein, 19 aa, Artificial Sequence
  Selected library sequence
GWWCYRNDSGPKPFHCRIK

SEQ ID NO: 20 - protein, 19 aa, Artificial Sequence
  Selected library sequence
EGWCWFIDSGPWKTWCEKQ

SEQ ID NO: 21 - protein, 19 aa, Artificial Sequence
  Selected library sequence
FPKCKFDFSGPPWYQCNTK

SEQ ID NO: 22 - protein, 19 aa, Artificial Sequence
  Selected library sequence
RTTCHHQISGPHGKICVNY

SEQ ID NO: 23 - protein, 19 aa, Artificial Sequence
  Selected library sequence
SNKCDHYQSGPHGKICVNY

SEQ ID NO: 24 - protein, 19 aa, Artificial Sequence
  Selected library sequence
WQECTKVLSGPGQFECEYM

SEQ ID NO: 25 - protein, 19 aa, Artificial Sequence
  Selected library sequence
RLDCDKVFSGPYGRVCVKY

SEQ ID NO: 26 - protein, 19 aa, Artificial Sequence
  Selected library sequence
WQECTKVLSGPGQFSCVYG

SEQ ID NO: 27 - protein, 19 aa, Artificial Sequence
  Selected library sequence
RLDCDKVFSGPYGNVCVNY

SEQ ID NO: 28 - protein, 19 aa, Artificial Sequence
  Selected library sequence
RLDCDKVFSGPSMGTCKLQ

SEQ ID NO: 29 - protein, 19 aa, Artificial Sequence
  Selected library sequence
RTTCHHHISGPHGKICVNY

SEQ ID NO: 30 - protein, 19 aa, Artificial Sequence
  Selected library sequence
QFGCEHIMSGPHGKICVNY

SEQ ID NO: 31 - protein, 19 aa, Artificial Sequence
  Selected library sequence
PVHCSHTISGPHGKICVNY

SEQ ID NO: 32 - protein, 19 aa, Artificial Sequence
  Selected library sequence
SVTCHFQMSGPHGKICVNY

SEQ ID NO: 33 - protein, 19 aa, Artificial Sequence
  Selected library sequence
PRGCQHMISGPHGKICVNY

SEQ ID NO: 34 - protein, 19 aa, Artificial Sequence
  Selected library sequence
RTTCHHQISGPHGQICVNY

SEQ ID NO: 35 - protein, 19 aa, Artificial Sequence
  Selected library sequence
WTICHMELSGPHGKICVNY

SEQ ID NO: 36 - protein, 19 aa, Artificial Sequence
  Selected library sequence
FITCALWLSGPHGKICVNY

SEQ ID NO: 37 - protein, 19 aa, Artificial Sequence
  Selected library sequence
MGGCDFSFSGPHGKICVNY

SEQ ID NO: 38 - protein, 19 aa, Artificial Sequence
  Selected library sequence
KDWCHTTFSGPHGKICVNY

SEQ ID NO: 39 - protein, 19 aa, Artificial Sequence
  Selected library sequence
AWGCDNMMSGPHGKICVNY

SEQ ID NO: 40 - protein, 19 aa, Artificial Sequence
  Selected library sequence
SNKCDHIMSGPHGKICVNY

SEQ ID NO: 41 - protein, 19 aa, Artificial Sequence
  Selected library sequence
SNKCDHYQSGPFGDICVMY

SEQ ID NO: 42 - protein, 19 aa, Artificial Sequence
  Selected library sequence
SNKCDHYQSGPFGDVCVSY

SEQ ID NO: 43 - protein, 19 aa, Artificial Sequence
  Selected library sequence
SNKCDHYQSGPFGDICVSY

SEQ ID NO: 44 - protein, 19 aa, Artificial Sequence
  Selected library sequence
RTTCHHQISGPFGPVCVNY

SEQ ID NO: 45 - protein, 19 aa, Artificial Sequence
  Selected library sequence
RTTCHHQISGPYGDICVKY

SEQ ID NO: 46 - protein, 19 aa, Artificial Sequence
  Selected library sequence
RYKCPRDLSGPPYGPCSPQ

SEQ ID NO: 47 - protein, 19 aa, Artificial Sequence
  Selected library sequence
WQECTKVLSGPGTFECSYE

SEQ ID NO: 48 - protein, 19 aa, Artificial Sequence
  Selected library sequence
SNKCDHYQSGPYGEVCFNY

SEQ ID NO: 49 - protein, 19 aa, Artificial Sequence
  Selected library sequence
RLDCDKVFSGPYGKVCVSY

SEQ ID NO: 50 - protein, 18 aa, Artificial Sequence
  Selected library sequence
RLDCDKVFSGPDTSCGSQ

SEQ ID NO: 51 - protein, 19 aa, Artificial Sequence
  Selected library sequence
RLDCDKVFSGPHGKICVRY

SEQ ID NO: 52 - protein, 19 aa, Artificial Sequence
  Selected library sequence
RVDCDKVISGPHGKICVNY

SEQ ID NO: 53 - protein, 19 aa, Artificial Sequence
  Selected library sequence
EFHCHHIMSGPHGKICVNY

SEQ ID NO: 54 - protein, 19 aa, Artificial Sequence
  Selected library sequence
HNRCDFKMSGPHGKICVNY

SEQ ID NO: 55 - protein, 19 aa, Artificial Sequence
  Selected library sequence
WQECTKVLSGPNSFECKYD

SEQ ID NO: 56 - protein, 19 aa, Artificial Sequence
  Selected library sequence
WDRCERQISGPGQFSCVYG

SEQ ID NO: 57 - DNA, 8195 bp, Artificial Sequence
  MANP vector
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattccatggcccgcccagatttctgtctcgagccaccatacactgggccctgcaaagcgcgcatcatccgctatttctacaatgctaaagcaggcctgtgccagacctttgtatacggtggttgccgtgctaagcgtaacaactttaaatcggccgaagattgcatgcgtacctgcggttctgcagatccttcatacattgaaggtcgtattgtcggtagcgccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaagatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgactgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 58 - DNA, 8171 bp, Artificial Sequence
  MANP vector
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattccatggcccgcccagatttctgtctcgagccaccaactgggccctgcaaagcgcgcatcatccgctatttctacaatgctaaagcaggcctgtgccagacctttgtatacggtggttgccgtgctaagcgtaacaactttaaatcggccgaagattgcatgcgtacctgcggttctgcagatccttcatacattgaaggtcgtattgtcggtagcgccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcacaaaacttattaccagtacactcctgtatcatcaaaagccatgtatgacgcttactggaacggtaaattcagagactgcgctttccattctggctttaatgaagatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttctttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgactgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 59 - protein, 124 aa, Artificial Sequence
  Signal sequence
MKKLLFAIPLVVPFYSMARPDFCLEPPYTGPCKARIIRYFYNAKAGLCQTFVYGGCRAKRNNFKCAEDCMATCGSADPSYIEGRIVGSAAETVESCLAKPHTENSFTNVWKDDKTLDRYANYEG

SEQ ID NO: 60 - protein, 30 aa, Artificial Sequence
  VARIANT 8, 9, 10, 12, 13, 14, 18, 19, 20, 22, 23, 24: Xaa = Any amino acid except cys
SMAAESGXXXCXXXSGPXXXCXXXGSESAD

SEQ ID NO: 61 - DNA, 99 bp, Artificial Sequence
  misc_feature 22, 23, 25, 26, 28, 29, 34, 35, 37, 38, 40, 41, 52, 53,55,56, 58, 59, 64, 65, 67, 68, 70, 71: n = any nucleotide
  misc_feature 24, 27, 30, 36, 39, 42, 54, 57, 60, 66, 69, 72: k = g or t
tccatggctgctgagtccggtnnknnknnktgcnnknnknnktccggtccgnnknnknnktgtnnknnknnkggtagcgagtctgcagaccactgcgac

SEQ ID NO: 62 - protein, 32 aa, Artificial Sequence
  Vector sequence
  VARIANT 8, 9, 10, 12, 13, 14, 15, 19, 20, 21, 22, 24, 25, 26: Xaa = Any amino acid except cys
SMAAESGXXXCXXXXSGPXXXXCXXXGSESAD

SEQ ID NO: 63 - DNA, 105 bp, Artificial Sequence
  Vector sequence
  misc_feature 22, 23, 25, 26, 28, 29, 34, 35, 37, 38, 40, 41, 43,44, 55,56, 58, 59, 61, 62, 64, 65, 70, 71, 73, 74, 76, 77: n = any nucleotide
  misc_feature 24, 27, 30, 36, 39, 42, 45, 57, 60, 63, 66, 72, 75, 78: k = g or t
tccatggctgctgagtccggtnnknnknnktgcnnknnknnknnktccggtccgnnknnknnknnktgtnnknnknnkggtagcgagtctgcagaccactgcgac

SEQ ID NO: 64 - protein, 29 aa, Artificial Sequence
  Vector sequence
  VARIANT 6, 7, 8, 9, 10, 11, 12, 13, 17, 18, 19, 20, 21, 22, 23,24: Xaa = Any amino acid except cys
TMAAEXXXXXXXXPSGXXXXXXXXGSESA

SEQ ID NO: 65 - DNA, 90 bp, Artificial Sequence
  Vector sequence
  misc_feature 16, 17, 19, 20, 22, 23, 25, 26, 28, 29, 31, 32, 34, 35,37,38, 49, 50, 52, 53, 55, 56, 58, 59, 61, 62, 64, 65: n = any nucleotide
  misc_feature 67, 68, 70, 71: n = any nucleotide
  misc_feature 18, 21, 24, 27, 30, 33, 36, 39, 51, 54, 57, 60, 63, 66,69,72: k = g or t
accatggctgctgagnnknnknnknnknnknnknnknnkccctcaggtnnknnknnknnknnknnknnknnkggtagcgagtctgcagtc